(19) United States
(12) Patent Application Publication
(10) Pub. No.: US 2003/0157481 A1
(43) Pub. Date: Aug. 21, 2003

(54) Diagnosing and treating cancer cells using T-HR mutants and their targets

(76) Inventors: Thomas L. Benjamin, Cambridge, MA (US); Yu Tian, Jamaica Plain, MA (US)

Correspondence Address: CLARK & ELBING LLP, 101 FEDERAL STREET, BOSTON, MA, 02110, US

(21) Appl. No.: 10/316,532

(22) Filed: Dec. 10, 2002

Related U.S. Application Data
(60) Provisional application No. 60/339,140, filed on Dec. 10, 2001.

(51) Int. Cl. C12Q 1/70; C12Q 1/68

(52) U.S. Cl. 435/5; 435/6

(57) ABSTRACT

The invention features a novel T-HR mutant virus, new primary cellular targets for DNA tumor viruses, such as Taz, as well as cellular factors, such as Death Inducer with SAP Domain (DIS) polypeptides, that interact with these primary targets. In addition, the invention encompasses DIS nucleic acid and amino acid sequences. The compounds described herein may be used in methods for diagnosing and treating patients having proliferative disorders, such as cancers.

### CROSS REFERENCE TO RELATED APPLICATIONS

This application claims priority to U.S. provisional application serial No. 60/339,140, filed on Dec. 10, 2001, which is hereby incorporated by reference.

### STATEMENT AS TO FEDERALLY SPONSORED RESEARCH

The present research was supported by grants from the National Cancer Institute (Numbers R35 44343 and CA92520). The Government has certain rights to this invention.

### FIELD OF THE INVENTION

The field of the invention is regulation of cellular proliferation.

### BACKGROUND OF THE INVENTION

Transforming genes of DNA tumor viruses perform essential functions in virus growth, acting largely as proto-oncogene activators or tumor suppressor gene inactivators. The isolation and characterization of mutant viruses that are able to propagate in cells containing a mutation in known proto-oncogene or tumor suppressor genes has been useful in identifying and studying the viral equivalents or interactors of these genes. The transforming gene of the highly oncogenic murine polyoma virus was identified through studies of host range mutants isolated using polyoma transformed 3T3 cells as the permissive host and normal 3T3 cells as the non-permissive host. This approach requires expression of a known viral protein by the permissive host, since it is based on the idea of complementation between cell-associated wild-type viral genes and an infecting virus mutant. In addition to its use with polyoma virus, the complementation approach has also been successfully used with other oncogenic DNA viruses, e.g., with 293 cells expressing adenovirus E1A/E1B genes and COS cells expressing the SV40 large T antigen. Complementing cell lines have also been used in other systems to propagate specifically defective virus mutants for vaccine development and other purposes. By design, these types of systems rely on permissive hosts constructed with known gain-of-function mutations and are only applicable to the study of mutations in known viral genes, as well as to viruses with known mutations, since the host cell must express a functional version of the mutant viral protein.

Unlike the systems described above, we developed a general, unbiased method for identifying new genes involved in the pre-disposition for, or progression of, cancer or other proliferative disorders using tumor host range (T-HR) mutant viruses. We previously used this system to identify a novel gene, sal2, which is found in nuclei of germinal epithelial cells from normal human ovary but is missing or altered in ovarian carcinomas derived from these cells (Li et al., Proc. Natl. Acad. Sci. USA 98:14,619-14,624, 2001 and U.S. patent application Ser. No. 09/812,633, which are hereby incorporated by reference). In view of the above results, we showed that T-HR viruses may be used to identify new genes involved in proliferative disorders.

While a number of genes are known to be involved in the progression towards cancer, there is a significant need for the identification and characterization of new genes involved in the pre-disposition for, or progression of, cancer or other proliferative disorders. Furthermore, methods for diagnosing and treating patients with mutations in such genes would greatly aid in the management of cancer.

### SUMMARY OF THE INVENTION

The invention features a novel T-HR mutant virus, new primary cellular targets for DNA tumor viruses, such as Taz, as well as cellular factors, such as Death Inducer with SAP Domain (DIS) polypeptides, that interact with these primary targets. In addition, the invention encompasses DIS nucleic acid and amino acid sequences. The compounds described herein may be used in methods for diagnosing and treating patients having proliferative disorders, such as cancers.

Accordingly, the first aspect of the invention features a method of determining the presence or absence of an alteration in the genetic material of a cell, for example, a cell from a mammal, such as a human. This method involves determining whether a cell can act as a permissive host for the replication and dissemination of a BMD-13 T-HR mutant virus, where the BMD-13 T-HR mutant virus is capable of replicating and disseminating in an abnormally proliferating cell and not in a normal cell. In a desirable embodiment of this method, the presence of the alteration in the genetic material is indicative of an organism carrying this genetic alteration having, or being at an increased risk of developing, a proliferative disease.

In other embodiments of this aspect, the cell is determined to have an alteration in a Taz, a GAP SH3 binding protein, a nucleolin, a Vesicle Associated Protein 1, or a Death Inducer with SAP Domain nucleic acid sequence or polypeptide.

In addition, the BMD-13 T-HR mutant virus may contain an alteration (e.g., an Aspartic Acid to Asparagine substitution) at the second position of the amino acid sequence of any of the polyoma T antigens, or the BMD-13 T-HR mutant virus may contain a deletion of amino acids 2 to 4 of any of the polyoma T antigens.

In a second aspect, the invention features a method of killing an abnormally proliferating cell, for example, a mammalian cell, such as a human cell. This method involves contacting an abnormally proliferating cell with a T-HR mutant specific for a cell carrying a Taz, a GAP SH3 binding protein, a nucleolin, a Vesicle Associated Protein 1, or a Death Inducer with SAP Domain alteration, and allowing this T-HR mutant to lyse said cell.

In desirable embodiments of this aspect of the invention the T-HR mutant virus is a BMD-13 T-HR mutant virus, for example, one that contains an alteration at the second position of the amino acid sequence of any of the polyoma T antigens. Such an alteration may be, for example, an Aspartic Acid to Asparagine substitution or the deletion of amino acids 2 to 4 of any of the polyoma T antigens. In addition, the T-HR mutant may be administered in a pharmaceutically acceptable carrier.

In another desirable embodiment the T-HR mutant virus is a mutant of a simian virus 40, human polyoma virus, herpes virus, primate adenoviruses, parvovirus, or a papilloma virus.

In a third aspect, the invention features a BMD-13 T-HR mutant virus, for example, one that contains an alteration at the second position of the amino acid sequence of any of the polyoma T antigens. This alteration may be an Aspartic Acid to Asparagine substitution or a deletion of amino acids 2 to 4 of any of the polyoma T antigens.

In a fourth aspect, the invention features an isolated nucleic acid encoding a Death Inducer with SAP Domain amino acid sequence, where this Death Inducer with SAP Domain amino acid sequence is at least 30% identical to the amino acid sequence of SEQ ID NO:2 or SEQ ID NO:4 and induces DNA condensation and apoptosis in a mammalian cell. However, this Death Inducer with SAP Domain amino acid sequence may also include the amino acid sequence of SEQ ID NO:2 or SEQ ID NO:4. In addition, the nucleic acid encoding the Death Inducer with SAP Domain amino acid may include the nucleic acid sequence of SEQ ID NO:1 or SEQ ID NO:3.

In a fifth aspect, the invention features a method of killing an abnormally proliferating cell. This method involves contacting the abnormally proliferating cell with a Death Inducer with SAP Domain nucleic acid sequence, where this contacting results in the expression of a DIS polypeptide in the abnormally proliferating cell. The Death Inducer with SAP Domain nucleic acid sequence may include, for example, the nucleic acid sequence of SEQ ID NO:1 or SEQ ID NO:3. In addition, the abnormally proliferating cell may be an endometrial, prostate, or ovarian cell.

In the sixth aspect, the invention features a method of identifying a mammal, for example, a human, having or at increased risk of acquiring a proliferative disease. This method includes the step of determining whether there is a loss of heterozygosity in a Death Inducer with SAP Domain nucleic acid of the mammal, where a loss of heterozygosity in a Death Inducer with SAP Domain nucleic acid is indicative of the mammal having, or being at risk of acquiring a proliferative disease. In a desirable embodiment, this method is used to identify a mammal having a proliferative disease and in another desirable embodiment, this method is used to identify a mammal at increased risk of acquiring a proliferative disease. In further desirable embodiments of this aspect, the method involves the use of polymerase chain reaction (PCR) amplification, single nucleotide polymorphism (SNP) determination, restriction fragment length polymorphism (RFLP) analysis, hybridization analysis, or mismatch detection analysis.

A seventh aspect of the invention features a method of decreasing proliferation of an abnormally proliferating cell. This method includes the step of contacting the abnormally proliferating cell with a Taz nucleic acid sequence, where this contacting results in the expression of a Taz polypeptide having wild-type activity in the abnormally proliferating cell.

In an eighth aspect, the invention features a method of decreasing virus, for example, tumor virus, replication and dissemination. This method includes the step of contacting a cell infected with a virus with a T-HR mutant target gene nucleic acid sequence, where this contacting results in the expression of a T-HR mutant target gene encoded polypeptide in the cell infected with the virus and prevents the virus from replicating and disseminating, or, for instance, from replicating or disseminating. For example, the virus may be a DNA tumor virus. In addition, in desirable embodiments, the T-HR mutant target gene nucleic acid sequence may be a Taz, a GAP SH3 binding protein, a nucleolin, a Vesicle Associated Protein 1, or a Death Inducer with SAP Domain nucleic acid sequence, such as the Death Inducer with SAP Domain nucleic acid sequence of SEQ ID NO:1 or SEQ ID NO:3.

The ninth aspect of the invention features a knockout mouse including a knockout mutation in a genomic Death Inducer with SAP Domain nucleic acid sequence and the tenth aspect features a transgenic mouse whose genome includes a nucleic acid construct containing a Death Inducer with SAP Domain nucleic acid sequence which is operably linked to transcriptional regulatory elements, for example, a tissue specific promoter, and encodes a Death Inducer with SAP Domain polypeptide. In one embodiment of the tenth aspect, the Death Inducer with SAP Domain polypeptide is mutant.

A eleventh aspect of the invention features a cell line derived from cells isolated from the transgenic mouse of the tenth aspect of the invention.

In a twelfth aspect, the invention features a method of identifying a compound which modulates cell proliferation. This method involves: a) exposing a cell or a cell extract to a test compound, and b) measuring whether the test compound modulates Taz, Nucleolin, Vesicle Associated Protein 1, or Death Inducer with SAP Domain levels, relative to Taz, Nucleolin, Vesicle Associated Protein 1, or Death Inducer with SAP Domain levels in a cell or cell extract not exposed to the test compound. In desirable embodiments of this aspect of the invention Taz, Nucleolin, Vesicle Associated Protein 1, or Death Inducer with SAP Domain is a Taz, Nucleolin, Vesicle Associated Protein 1, or Death Inducer with SAP Domain polypeptide or a Taz, Nucleolin, Vesicle Associated Protein 1, or Death Inducer with SAP Domain nucleic acid. In additional desirable embodiments, Taz, Nucleolin, Vesicle Associated Protein 1, or Death Inducer with SAP Domain polypeptide or nucleic acid levels may be measured. In further desirable embodiments, the compound identified using the method of the twelfth aspect of the invention may be used to treat a proliferative disease, for example, one that is due to a proliferative disease-associated alteration in a Taz, Nucleolin, Vesicle Associated Protein 1, or Death Inducer with SAP Domain nucleic acid sequence. Desirable examples of proliferative diseases include cancers such as leukemias or ovarian cancer.

Definitions

“Tumor host range mutant virus (T-HR mutant),” as used herein, refers to a virus that has a reduced ability to replicate and disseminate in a normal cell, relative to the replication of a wild-type virus in the same type of cell, but is able to replicate and disseminate in an abnormally proliferating cell. The abnormally proliferating cell may, for example, be a transformed or tumor-derived cell with one or more mutations in a gene or genes involved in the regulation of cell growth, of the cell cycle, or of programmed cell death (e.g., apoptosis). These genes include, for example, tumor suppressor genes and proto-oncogenes, but any cellular gene that a virus must inactive or activate, either directly or indirectly, to grow is also included. Adenoviruses having mutations affecting interactions with the p53 and retinoblastoma genes are specifically excluded. In one embodiment the virus has a mammalian (e.g., rodent or primate) host range. In a more desirable embodiment, the virus has a human host range. A T-HR mutant virus may be, for example, a mutant simian virus 40, human polyoma virus, parvovirus, papilloma virus, herpes virus, or a primate adenovirus. However, any virus that needs to overcome a cell cycle checkpoint or affect signal transduction to propagate may be a T-HR mutant virus.

By “tumor virus,” as used herein is meant a virus that has a mammalian (e.g., rodent, primate, or human) host range that can, as a consequence of infecting a cell, transform a normal cell into an abnormally proliferating cell. A “tumor virus” as used herein may, for example, be a DNA tumor virus or an RNA tumor virus. Examples of “tumor viruses” include simian virus 40, murine or human polyoma virus, parvovirus, papilloma virus, herpes virus, and primate adenovirus.

“BMD-13 T-HR mutant virus,” as used herein, refers to a T-HR mutant virus, containing an alteration in the common N-terminus of any of the polyoma T antigens. For example, this alteration may be a single amino acid substitution in the second position of any of the polyoma T antigens, e.g., an Aspartic Acid to Asparagine (D to N) substitution, or the deletion of amino acids 2-4 of any of the polyoma T antigens. In addition, the alteration may be present in all polyoma T antigens. A “BMD-13 T-HR mutant virus” is able to replicate and disseminate in abnormally proliferating cells, for example, BNL cells, a carcinogen-induced mouse liver cell line, but has a reduced ability to replicate and disseminate in primary, normal cells, for example, baby mouse kidney (BMK) cells. In addition, the abnormally proliferating cells may have a proliferative disease-associated alteration in a Taz nucleic acid sequence, in a nucleic acid sequence affecting expression of a protein that a Taz gene product interacts with, or in a nucleic acid sequence encoding a component of a signaling pathway involving Taz. Examples of proteins that interact with Taz gene products include ras-GTPase-activating protein SH3-domain binding protein (G3BP), Nucleolin, Vesicle Associated Protein 1, and Death Inducer with SAP domain (DIS).

By “a T-HR mutant target gene,” as used herein is meant any cellular gene that a virus must inactivate or activate, either directly or indirectly, to replicate and disseminate. Examples of “T-HR mutant target genes” include Taz, ras-GTPase-activating protein SH3-domain binding protein (G3BP), Nucleolin, Vesicle Associated Protein 1, or Death Inducer with SAP domain (DIS) nucleic acid sequence. In one embodiment, these nucleic acid sequences are mammalian. In more desirable embodiments the nucleic acid sequences are murine or human nucleic acid sequences.

By a “Taz polypeptide having wild-type activity” is meant a Taz polypeptide that reduces the ability of a BMD-13 T-HR mutant to replicate and disseminate in a cell. The reduction in the ability of a BMD-13 T-HR mutant to replicate and disseminate may be measured by determining the number of cells infected with the BMD-13 T-HR mutant that survive in presence of a “Taz polypeptide having wild-type activity” in comparison to the number of cells infected with the BMD-13 T-HR mutant that survive in the absence of a “Taz polypeptide having wild-type activity,” where an increase in the number of cells surviving is indicative of a reduction in ability of the BMD-13 T-HR mutant to replicate and disseminate. In one embodiment, the reduction in the ability of a BMD-13 T-HR to replicate and disseminate is at least 25%. In more desirable embodiments, the reduction in the ability of a BMD-13 T-HR mutant to replicate and disseminate is at least 50%, 75%, 80%, 90%, or 95%. However, a “Taz polypeptide having wild-type activity” may also completely block the ability of a BMD-13 T-HR mutant to replicate and disseminate. In addition, a “Taz polypeptide having wild-type activity” may have further functions in a cell besides reducing the ability of a BMD-13 T-HR mutant virus to replicate and disseminate.

By a “DIS nucleic acid sequence” or “Death Inducer with SAP domain nucleic acid sequence,” as used herein is meant a nucleic acid sequence that is at least 40%, 50%, 60%, 70%, 75%, 80%, 85%, 90%, 95%, or 99% identical to a nucleic acid sequence provided of SEQ ID NO:1 or SEQ ID NO:3 over a region comprising at least 200, 300, 500, 750, 1000, 1500, 2000, 2500, 3000, or 3500 contiguous nucleotides. In addition, a “DIS nucleic acid sequence” may be identical to the nucleic acid sequence of SEQ ID NO:1 or SEQ ID NO:3. In desirable embodiments, a “DIS nucleic acid sequence” is a human or a mouse DIS nucleic acid sequence that is at least 75%, 80%, 85%, 90%, or 95% identical to the human DIS nucleic acid sequence of SEQ ID NO:3, or to the murine DIS nucleic acid sequence of SEQ ID NO:1, over a region encompassing at least 1000, 2000, 3000, or 3500 contiguous nucleotides, and encodes a protein which induces DNA condensation and apoptosis in mammalian cells.

By a “DIS polypeptide,” a “Death Inducer with SAP domain polypeptide,” a “DIS amino acid sequence,” or a “Death Inducer with SAP domain amino acid sequence,” as used herein is meant an amino acid sequence that is at least 30%, 40%, 50%, 60%, 70%, 75%, 80%, 85%, 90%, 95%, or 99% identical to the amino acid sequence of SEQ ID NO:2 or SEQ ID NO:4 over a region comprising at least 50, 75, 100, 200, 300, 500, 700, 900, or 1200 contiguous amino acids. In addition, a “DIS polypeptide” may be identical to the amino acid sequence of SEQ ID NO:2 or SEQ ID NO:4. In desirable embodiments, a “Death Inducer with SAP domain (DIS) polypeptide” or a “Death Inducer with SAP domain (DIS) amino acid sequence” is a human or a mouse DIS polypeptide or amino acid sequence that is at least 30%, 50%, 60%, 70%, 80%, 90%, or 95% identical to the human DIS amino acid sequence of SEQ ID NO:4, or the mouse DIS amino acid sequence of SEQ ID NO:2, over a region encompassing 500, 700, 900, or 1200 contiguous amino acids, and induces DNA condensation and apoptosis in mammalian cells.

By a “Taz nucleic acid sequence,” as used herein is meant a nucleic acid sequence that is at least 40%, 50%, 60%, 70%, 75%, 80%, 85%, 90%, 95%, or 99% identical to the nucleic acid sequence of GenBank Accession No. AI317016 over a region comprising at least 100, 200, 300, 400, or 500 contiguous nucleotides. In addition, a “Taz nucleic acid sequence” may be identical to the nucleic acid sequence of GenBank Accession No. AI317016. In desirable embodiments, a “Taz nucleic acid sequence” is a human or a mouse Taz nucleic acid sequence. Furthermore, a “Taz nucleic acid sequence” may also include upstream regulatory sequences.

By a “Taz polypeptide” or a “Taz amino acid sequence,” as used herein is meant an amino acid sequence that is at least 30%, 40%, 50%, 60%, 70%, 75%, 80%, 85%, 90%, 95%, or 99% identical to the amino acid sequence encoded by GenBank Accession No. AI317016 over a region comprising at least 50, 75, 100, or 150 contiguous amino acids. In addition, a “Taz polypeptide” or a “Taz amino acid sequence” may be identical to the amino acid sequence encoded by GenBank Accession No. AI317016. In desirable embodiments, a “Taz polypeptide” or a “Taz amino acid sequence” is a human or a mouse Taz polypeptide or amino acid sequence.

By a “GAP SH3 binding protein nucleic acid sequence” or “G3BP nucleic acid sequence,” as used herein is meant a nucleic acid sequence that is at least 40%, 50%, 60%, 70%, 75%, 80%, 85%, 90%, 95%, or 99% identical to a nucleic acid sequence encoding the amino acid sequence of GenBank Accession Nos. NP_{—}038744 or 7305075 over a region comprising at least 300, 500, 750, 1000, or 1200 contiguous nucleotides. In addition, a “G3BP nucleic acid sequence” may be identical to a nucleic acid sequence encoding the amino acid sequence of GenBank Accession Nos. NP_{—}038744 or 7305075. In desirable embodiments, a “GAP SH3 binding protein nucleic acid sequence” or “G3BP nucleic acid sequence” is a human or a mouse GAP SH3 binding protein nucleic acid sequence.

By a “GAP SH3 binding protein polypeptide,” a “G3BP polypeptide,” a “GAP SH3 binding protein amino acid sequence,” or a “G3BP amino acid sequence,” as used herein is meant an amino acid sequence that is at least 30%, 40%, 50%, 60%, 70%, 75%, 80%, 85%, 90%, 95%, or 99% identical to the amino acid sequence provided in GenBank Accession Nos. NP_{—}038744 or 7305075 over a region comprising at least 50, 75, 100, 200, 300, or 400 contiguous amino acids. In addition, a “G3BP polypeptide” or a “G3BP amino acid sequence” may be identical to the amino acid sequence provided in GenBank Accession Nos. NP_{—}038744 or 7305075. In desirable embodiments, a “G3BP polypeptide” or a “G3BP amino acid sequence” is a human or a mouse G3BP polypeptide or amino acid sequence.

By a “Nucleolin nucleic acid sequence,” as used herein is meant a nucleic acid sequence that is at least 40%, 50%, 60%, 70%, 75%, 80%, 85%, 90%, 95%, or 99% identical to a nucleic acid sequence encoding the amino acid sequence of GenBank Accession Nos. AAH05460 or 13529464 over a region comprising at least 300, 500, 750, 1000, 1500, or 2000 contiguous nucleotides. In addition, a “Nucleolin nucleic acid sequence” may be identical to a nucleic acid sequence encoding the amino acid sequence of GenBank Accession Nos. AAH05460 or 13529464. In desirable embodiments, a “Nucleolin nucleic acid sequence” is a human or a mouse Nucleolin nucleic acid sequence.

By a “Nucleolin polypeptide” or a “Nucleolin amino acid sequence,” as used herein is meant an amino acid sequence that is at least 30%, 40%, 50%, 60%, 70%, 75%, 80%, 85%, 90%, 95%, or 99% identical to the amino acid sequence provided in GenBank Accession Nos. AAH05460 or 13529464 over a region comprising at least 50, 75, 100, 200, 300, 400, 500, 600, or 700 contiguous amino acids. In addition, a “Nucleolin polypeptide” or a “Nucleolin amino acid sequence” may be identical to the amino acid sequence provided in GenBank Accession Nos. AAH05460 or 13529464. In desirable embodiments, a “Nucleolin polypeptide” or a “Nucleolin amino acid sequence” is a human or a mouse Nucleolin polypeptide or amino acid sequence.

By a “Vesicle Associated Protein 1 nucleic acid sequence,” as used herein is meant a nucleic acid sequence that is at least 40%, 50%, 60%, 70%, 75%, 80%, 85%, 90%, 95%, or 99% identical to a nucleic acid sequence encoding the amino acid sequence of GenBank Accession Nos. T14150 or 7514116 over a region comprising at least 300, 500, 750, 1000, 1500, 2000, 2500, 3000, or 3500 contiguous nucleotides. In addition, a “Vesicle Associated Protein 1 nucleic acid sequence” may be identical to a nucleic acid sequence encoding the amino acid sequence of GenBank Accession Nos. T14150 or 7514116. In desirable embodiments, a “Vesicle Associated Protein 1 nucleic acid sequence” is a human or a mouse Vesicle Associated Protein 1 nucleic acid sequence.

By a “Vesicle Associated Protein 1 polypeptide” or a “Vesicle Associated Protein 1 amino acid sequence,” as used herein is meant an amino acid sequence that is at least 30%, 40%, 50%, 60%, 70%, 75%, 80%, 85%, 90%, 95%, or 99% identical to the amino acid sequence provided in GenBank Accession Nos. T14150 or 7514116 over a region comprising at least 50, 75, 100, 200, 400, 600, 800, 1000, or 1200 contiguous amino acids. In addition, a “Vesicle Associated Protein 1 polypeptide” or a “Vesicle Associated Protein 1 amino acid sequence” may be identical to the amino acid sequence provided in GenBank Accession Nos. T14150 or 7514116. In desirable embodiments, a “Vesicle Associated Protein 1 polypeptide” or a “Vesicle Associated Protein 1 amino acid sequence” is a human or a mouse Vesicle Associated Protein 1 polypeptide or amino acid sequence.

By “reduced ability to replicate and disseminate,” as used herein is meant a reduction in the ability of a mutant virus, for example, a mutant DNA tumor virus, to replicate and disseminate, relative to a wild-type virus in the same type of cell, of at least 50%, 60%, 70%, 80%, 90%, 95%, 99%, or the complete inability to replicate or disseminate. In one desirable embodiment, the ability to replicate and disseminate is reduced by at least 90%. In more desirable embodiments, the ability to replicate and disseminate is reduced by at least 95% or 99%.

“Cancer susceptibility gene,” as used herein, refers to any gene that, when altered, increases the likelihood that the organism carrying the gene will develop a proliferative disorder during its lifetime. Examples of such genes include proto-oncogenes, tumor suppressor genes, and genes involved in the regulation of cell growth, the cell cycle, checkpoint controls, and apoptosis. In desirable embodiments, a cancer susceptibility gene is a Taz, a Death Inducer with SAP domain, a ras-GTPase-activating protein SH3-domain binding protein (G3BP), a Nucleolin, or a Vesicle Associated Protein 1 nucleic acid sequence.

“Proliferative disease,” as used herein, refers to any disorder that results in the abnormal proliferation of a cell. Specific examples of proliferative diseases are various types of cancer, such as leukemia and liver cancer. However, proliferative diseases may also be the result of the cell becoming infected with a transforming virus.

“Proliferative disease-associated alteration,” as used herein, refers to any genetic change within a differentiated cell that results in the abnormal proliferation of a cell. In one desirable embodiment, such a genetic change correlates with a statistically significant (e.g., a p-value less than or equal to 0.05) increase in the risk of acquiring a proliferative disease. Examples of such genetic changes include mutations in genes involved in the regulation of the cell cycle, of growth control, or of apoptosis and can further include mutations in tumor suppressor genes and proto-oncogenes.

“Abnormal proliferation,” as used herein, refers to a cell undergoing cell division under inappropriate conditions. For example, a cell may be undergoing “abnormal proliferation” if the cell normally does not undergo cell division or if the cell does not respond to normal checkpoint controls.

By a compound that “modulates the protein level” or “modulates the nucleic acid level” is meant a compound that increases or decreases protein or nucleic acid level of a specific protein or nucleic acid in a cell or a cell extract. For example, such a compound may increase or decrease RNA stability, transcription, translation, or protein degradation. It will be appreciated that the degree of modulation provided by a modulatory compound in a given assay will vary, but that one skilled in the art can determine the statistically significant change (e.g., a p-value≦0.05) in the level of the specific protein or nucleic acid affected by a modulatory compound. In desirable embodiments, the protein or nucleic acid is a Taz, ras-GTPase-activating protein SH3-domain binding protein (G3BP), Nucleolin, Vesicle Associated Protein 1, or Death Inducer with SAP domain (DIS) amino acid or nucleic acid sequence.

“Alteration,” when used herein in reference to a gene, refers to a change in the coding or regulatory nucleic acid sequence or a modification of the nucleic acid sequence, for example, DNA methylation of the promoter region. The change in the coding or regulatory nucleic acid sequence may include, for example, an insertion, a deletion, or a substitution of one or more nucleic acids, as well as an inversion or a duplication. “Alteration,” when used herein in reference to a polypeptide refers to a change in the amino acid sequence. Such a change may be, for example, a substitution, a deletion, or an insertion.

“Genetic lesion,” as used herein, refers to a nucleic acid change. Examples of such a change include a single nucleic acid change as well as a deletion or an insertion of one or more nucleic acid. However, a genetic lesion can also include a duplication or an inversion. In addition, a genetic lesion may be a naturally-occurring polymorphism, for example, one that predisposes an organism carrying the polymorphism to acquiring a proliferative disease.

“Loss of heterozygosity,” as used herein, refers to a nucleic acid sequence that is homozygous for the same locus on a chromosome. For example, the normal copy of a gene is lost and both copies of the gene in a cell are mutant. A loss of heterozygosity may occur due to a structural deletion in the normal gene in the chromosome carrying this gene. Alternatively, a loss of heterozygosity may be due to recombination between the mutant and the normal gene, followed by formation of a daughter cell homozygous for the deleted or inactivated (i.e., mutant) gene. A loss of hetetozygosity may also result from a loss of the chromosome with the normal gene and a duplication of the chromosome with the mutant gene. A loss of heterozygosity may be determined by standard methods in the art, for example, by using Southern blots, by sequencing, or by PCR analysis (See, for example, Debelenko et al., Hum. Mol. Genet. 6:2285-2290, 1997; and Emmert-Buck et al., Cancer Res. 55:2959-2962, 1995).

“Polymorphism,” as used herein, refers to an alteration in a nucleic acid sequence, for example, a gene, that may result in a codon change.

“Modification of function,” as used herein, refers to a change in the function of the protein. Such a change can, for example, result in the partial or complete loss of function, but it can also result in a gain of function or a new function.

“Knockout,” as used herein, refers to an alteration in the sequence of a specific gene that results in a decrease of function of that gene. In desirable embodiments, the alteration results in undetectable or insignificant expression of the gene and in a complete or partial loss of function. Furthermore, the disruption may be conditional, e.g., dependent on the presence of tetracycline. Knockout animals may be homozygous or heterozygous for the gene of interest. In addition, the term knockout includes conditional knockouts, where the alteration of the target gene can occur, for example, as a result of exposure of the animal to a substance that promotes target gene alteration, introduction of an enzyme that promotes recombination at the target gene site (e.g., Cre in the Cre-lox system, or FLP in the FLP/FRT system), or any other method for directing target gene alteration.

“Operably linked,” as referred to herein, describes the functional relationship between nucleic acid sequences, for example, a promoter or enhancer sequence, and a gene to be expressed. However, since enhancers can exert their effect over long distances, they do not require close physical linkage in sequence to the gene whose transcription they affect.

The term “restriction fragment length polymorphism (RFLP) analysis,” as used herein, refers to a method of determining whether an organism carries a specific nucleic acid sequence, for example, a specific alteration in a gene. This method may involve, for example, amplification of a nucleic acid from the organism, followed by cleavage of the nucleic acid with an enzyme, such as a restriction enzyme, and visualizing the products of the cleavage reaction. Furthermore, the cleavage products may be compared to control reactions.

As used herein, “modulates proliferation” refers to any change in the proliferation of a cell, when compared to a control cell of the same type. For example, this term can be used to describe an increase or a decrease in the rate of cell division. In addition, a modulation of proliferation may refer to a normally quiescent cell entering into the cell cycle or a normally dividing cell ceasing to enter into the cell cycle.

“Measuring protein levels,” as used herein, includes any standard assay used in the art to either directly or indirectly determine protein levels. Such assays, for example, may include the use of an antibody, Western analysis, Bradford assays, and spectrophotometric assays.

“Measuring nucleic acid levels,” as used herein, includes any standard assay used in the art to either directly or indirectly determine nucleic acid levels. Such assays include, for example, hybridization analysis, gel electrophoresis, Northern blots, Southern blots, and spectrophotometric assays.

By a “substantially pure polypeptide” is meant a polypeptide that has been separated from components that naturally accompany it. Typically, the polypeptide is substantially pure when it is at least 60% or 70%, by weight, free from the proteins and naturally-occurring organic molecules with which it is naturally associated. In one desirable embodiment, the preparation is at least 75%, by weight, the desired polypeptide. In more desirable embodiments, the preparation is at least 80%, 85%, 90%, 95%, or 99%, by weight, the desired polypeptide. A substantially pure polypeptide may be obtained, for example, by extraction from a natural source (for example, a mammalian cell), by expression of a recombinant nucleic acid encoding a polypeptide, or by chemically synthesizing the protein. Purity can be measured by any appropriate method, for example, column chromatography, polyacrylamide gel electrophoresis, or by HPLC analysis.

By an “isolated DNA” or an “isolated nucleic acid” is meant a nucleic acid sequence that is free of the naturally-occurring nucleic acid sequences that flank the nucleic acid sequence of the invention in the organism. The term therefore includes, for example, a recombinant DNA that is incorporated into a vector, into an autonomously replicating plasmid or virus, into the genomic DNA of a prokaryote or eukaryote, or that exists as a separate molecule (for example, a cDNA or a genomic or cDNA fragment produced by PCR or restriction endonuclease digestion) independent of other sequences. It also includes a recombinant DNA that is part of a hybrid gene encoding additional polypeptide sequence.

By a “candidate compound” or “test compound” is meant a chemical, be it naturally-occurring or artificially-derived, that is surveyed for its ability to modulate cell proliferation, by employing one of the assay methods described herein. Candidate compounds may include, for example, peptides, polypeptides, synthetic organic molecules, naturally-occurring organic molecules, nucleic acid molecules, and components thereof.

By “high stringency hybridization conditions” is meant, for example, hybridization at approximately 42° C. in about 50% formamide, 0.1 mg/ml sheared salmon sperm DNA, 1% SDS, 2× SSC, 10% Dextran sulfate, a first wash at approximately 65° C. in about 2× SSC, 1% SDS, followed by a second wash at approximately 65° C. in about 0.1× SSC. Alternatively, “high stringency hybridization conditions” may include hybridization at approximately 42° C. in about 50% formamide, 0.1 mg/ml sheared salmon sperm DNA, 0.5% SDS, 5× SSPE, 1× Denhardt's, followed by two washes at room temperature in 2× SSC, 0.1% SDS, and two washes at between 55-60° C. in 0.2× SSC, 0.1% SDS.

Advantages

The use of T-HR mutant viruses has a particular advantage over standard chemotherapy treatments, and the like, in that it is targeted to cells with a proliferative disease. Therefore, one would expect this type of therapy to have fewer toxic side effects than the chemotherapeutic agents used today.

### BRIEF DESCRIPTION OF THE DRAWINGS

FIG. 1 is a schematic diagram of two embodiments of the BMD-13 T-HR mutant virus.

FIG. 2 is a schematic diagram of the murine Taz protein.

FIG. 3 is a picture of a Western blot showing that mTaz binds to the T antigens.

FIG. 4 is a picture of a Western blot showing that human Taz binds to the SV40 large T antigen in vivo.

FIG. 5 is a series of protein gels showing that amino acids 2-4 of murine Taz are essential for its interaction with middle (panel A) and small (panel B) T antigens.

FIG. 6 is a picture of a series of Western blots showing that Taz binds to PP2A and Src.

FIG. 7 is a picture of a protein gel showing that the phosphorylation state of murine Taz changes as a result of polyoma virus infection.

FIG. 8 is a series of scanned images showing the intracellular localization of murine Taz and how it is altered in response to wild-type or mutant polyoma virus infection.

FIG. 9 is a picture of a series of Western blots showing that the small T antigen increases binding of Taz to the large T antigen.

FIG. 10 is a non-limiting model of Taz function during polyoma virus infection.

FIG. 11 is the sense (SEQ ID NO:1) and the antisense strand of the murine DIS nucleic acid sequence as well as the corresponding amino acid sequence (SEQ ID NO:2).

FIG. 12 is the murine DIS nucleic acid sequence (SEQ ID NO:1).

FIG. 13 is the murine DIS nucleic acid sequence (SEQ ID NO:1) and the amino acid sequence encoded by the open reading frame of murine DIS (SEQ ID NO:2).

FIG. 14 is the sense (SEQ ID NO:3) and the antisense strand human DIS nucleic acid sequence as well as the corresponding amino acid sequence (SEQ ID NO:4).

FIG. 15 is the human DIS nucleic acid sequence (SEQ ID NO:3).

FIG. 16 is the human DIS nucleic acid sequence (SEQ ID NO:3) and the amino acid sequence encoded by the open reading frame of human DIS (SEQ ID NO:4).

FIG. 17 is a picture of a Southern blot showing that polyoma virus replication is inhibited by mTaz.

FIG. 18 is scanned image showing Tet induced TAZ expression (A) and a graph showing that TAZ inhibits origin replication (B).

FIG. 19 is a Southern blot (A) showing a loss of heterozygosity in DIS in ovarian tumors, and a Western blot (B) showing a lack of DIS expression in ovarian tumors.

FIG. 20 is a series of images showing that murine DIS induces apoptosis.

FIG. 21 is a series of Western blots showing that DIS, PARP, and LaminB are cleaved in BMK and HeLa cells upon induction of apoptosis by staurosporine (A), and that caspase-3 and caspase-8 inhibitors can inhibit DIS cleavage (B).

FIG. 22 is a series of schematic diagrams showing the location of several caspase-3 and caspase-8 cleavage sites in human (top) and murine (bottom) DIS.

FIG. 23 is a series of Western blots showing that DIS is sensitive to caspase-3 cleavage and that the first caspase-3 site, at amino acid 691 of human DIS and at amino acid 689 of murine DIS, is used for cleavage.

FIG. 24 is a schematic diagram showing the genomic organization of exon 2 of TAZ, and structure of the targeting vector used to generate TAZ knock out mice.

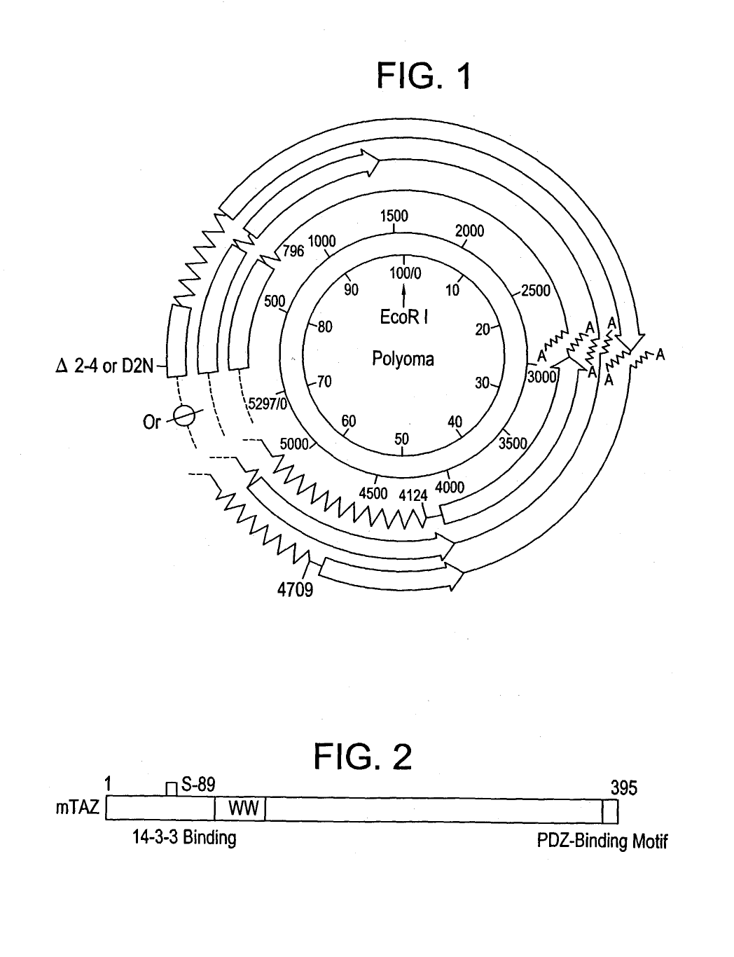

### DETAILED DESCRIPTION OF THE INVENTION

The present invention provides a novel T-HR virus, new targets for DNA tumor viruses, as well as novel nucleic acid and amino acid sequences, that may be used in diagnostic methods for identifying abnormally proliferating cells, or cells that have the potential to become abnormally proliferating cells, and in treatments aimed at selectively eliminating such cells.

Identification of the BMD-13 T-HR Mutant

We describe a tumor host range mutant virus (the BMD-13 T-HR mutant) that is capable of replicating in abnormally proliferating cells, but not in normal cells. In general, permissive hosts for T-HR mutants could fail to express, or carry a mutation in, the cellular target itself, or, alternatively, these hosts, or other cancer cells, may be defective in some interacting partner or effector of the target gene or in a gene which functions in the same pathway(s) as the target gene itself (Li et al., Proc. Natl. Acad. Sci. USA 98:14,619-14,624, 2001). Examples of the latter possibility are permissive hosts for polyoma or adenovirus mutants that are defective in binding pRb or p53, despite expressing these tumor suppressors themselves, as a result of defects in INK4A gene products which impinge on pRb and p53 (Freund et al., J. Virol. 68:7227-7234, 1994; Harvey and Levine, Genes & Dev. 5:2375-2385, 1991; Yang et al., Cancer Res. 61:5959-5963, 2001; McCormick, Oncogene 19:6670-6672, 2000; Linardopoulos et al., Cancer Res. 55:5168-5172, 1995).

The BMD-13 T-HR mutant virus has altered T antigens that are unable to interact with a cellular Taz protein. Since T-HR mutants generally have a mutation that causes a modification of function of the protein encoded by that gene and since these mutations typically lie in the transforming genes of the DNA tumor viruses and are usually activators of cellular proto-oncogenes or inactivators of tumor suppressor genes, the Taz protein is likely to be normally targeted by the viral transforming proteins. Accordingly, a normal Taz nucleic acid or protein, like other cellular targets of DNA tumor viruses, may be used as an anti-cancer or anti-viral agent. Furthermore, the BMD-13 T-HR mutant virus may be used to identify abnormally proliferating cells, or cells that have the potential to become abnormally proliferating cells, as well as to selectively kill such cells.

The general protocol used to isolate the BMD-13 T-HR mutant is outlined in Table 1 below.

### Table 1

### Tumor Host Range Mutants—Selection Procedure and Target Identification

I. Mutant Selection

1. Random mutagenesis of wild-type viral DNA

2. Amplification of the mutant virus by growth in tumor cells

3. Cloning by plaque isolation on tumor cells

4. Screening of plaque lysates for the absence of growth in normal cells

5. Molecular cloning and sequencing of the mutant viral DNA

II. Target Identification and Validation

6. Screening of a mouse embryo cDNA library in yeast with wild-type bait

7. Counterscreening positive clones for lack of interaction with mutant bait

8. Construction of complete cDNA expressing the target protein

9. Verification of viral protein-cellular target interactions in vitro and in vivo (e.g., T antigen-cellular protein interactions).

We used the tumor host range selection procedure to identify a T-HR mutant polyoma virus (BMD-13) that is able to replicate and disseminate in BNL cells (a carcinogen-induced mouse liver tumor derived cell line), but replicates and disseminates poorly in primary baby mouse kidney (BMK) cells. The inability of this virus to propagate on normal, primary cells is due to a single amino acid substitution in all polyoma T antigens (sT, mT, and lT). We determined that the BMD-13 T-HR mutant that we isolated encodes altered T antigen proteins that have an Aspartic Acid to Asparagine (D to N) substitution at the second position of the T antigen amino acid sequences.

To identify the binding target for these altered T antigens, we used a “flipped” bait yeast two-hybrid system. This method involved screening a mouse cDNA library made from 17 day-old embryos with a wild-type small T (sT) protein fused to the Gal4 DNA binding domain (sT-Gal4BD). Our screen yielded a single positive clone, mTaz (transcriptional co-activator with PDZ binding motif; Kanai et al., EMBO J. 19:6778-6791, 2000; GenBank Accession No. AI317016). As shown in FIG. 2, mTaz contains a 14-3-3 binding sequence, a PDZ binding motif, and a WW domain which recognizes proline-rich regions present in known transcription factors including PEBP2α and other members of the Runx family, alterations in which have been linked to human cancer.

PDZ domains were first identified in the post-synaptic density protein PSD95, in the Drosophila tumor suppressor protein Dlg1, and in the tight junction protein ZO-1, but now have been found in at least 600 proteins. Most PDZ domain containing proteins are membrane associated, but several have been shown to reside in, or transit to, the nucleus. 14-3-3 proteins, on the other hand, generally reside in the cytoplasm. These proteins form a large, ubiquitously expressed family found in virtually all organisms, including mammals, plants, yeast, and fungi. In general, 14-3-3 proteins bind to proteins that have been phosphorylated on serine or threonine residues.

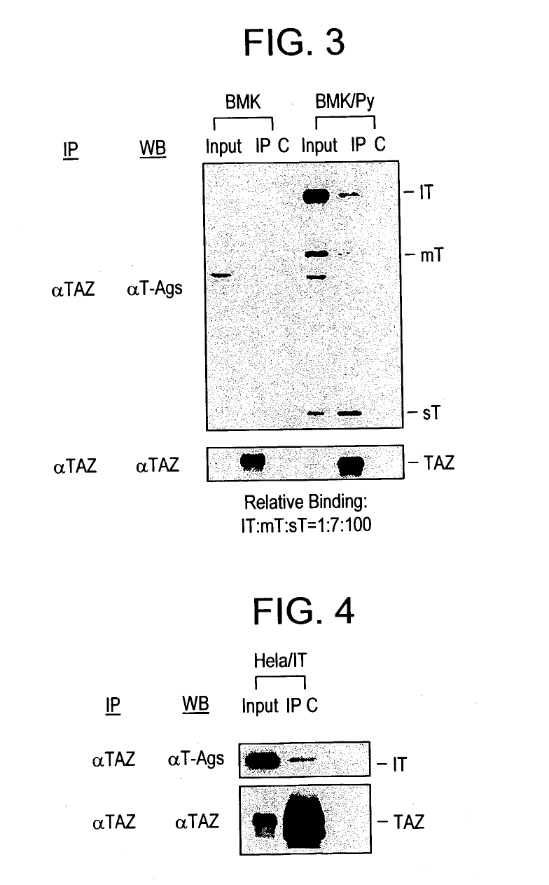

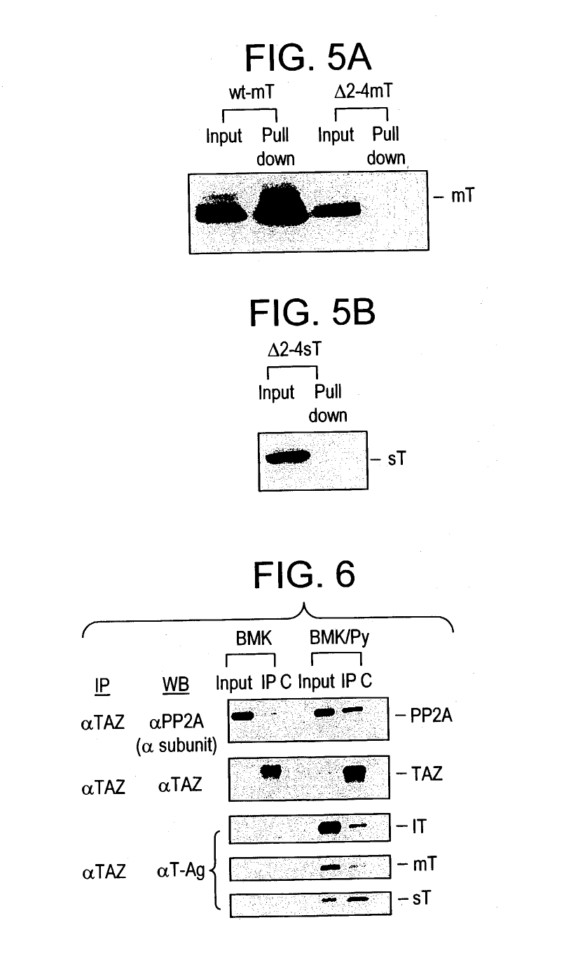

To verify the specificity of the interaction between T antigens and Taz, we immunoprecipitated Taz using a rabbit anti-Taz antibody from both uninfected and from wild-type polyoma virus infected BMK cells. We performed a Western analysis on the immunoprecipitated proteins and probed the Western blot with an anti-T antigen antibody. As is shown in FIG. 3, all three T antigens immunoprecipitated with Taz, but these proteins were present in different abundances. The relative binding efficiencies of murine Taz to the T antigens is large T: middle T: small T=1:7:100.

In addition, we confirmed the in vitro binding results by showing that human Taz interacts with the SV40 large T antigen in vivo. We transfected HeLa cells with both human Taz and SV40 large T antigen and used a rabbit anti-Taz antibody to immunoprecipitate Taz from extracts made from these cells. A Western blot of a protein gel on which these extracts were run shows that an anti-SV40 large T antigen antibody recognizes a protein of the appropriate size in the immunoprecipitated lane (FIG. 4).

Furthermore, we wanted to show that the region mutated in the BMD-13 T-HR virus interacts with Taz. We tested several N-terminal deletions and determined that a three amino acid deletion (Δ2-4) abolishes the interaction between Taz and small and middle T antigens. Not only do T antigens having the Δ2-4 fail to interact with Taz, but polyoma viruses carrying this deletion also fail to transform mammalian cells. We counted foci obtained by transfecting 1×10⁶ F111 rat fibroblast cells with 1 μg of virus genome and obtained more than 100 foci using a wild-type virus, but failed to obtain any foci with a Δ2-4 mutant polyoma virus. Accordingly, amino acids 2-4 of the T antigens are essential for transformation as well as for binding to Taz.

Proteins that Interact with TAZ

To further characterize the function of Taz, we looked at whether Taz binds to other proteins besides T antigens, in particular ones that are known to bind T antigens. In this regard, we performed another immunoprecipitation experiment and showed that Taz interacts with PP2A, a serine-threonine phosphatase known to associate with the large T antigen, as well as with c-src. Moreover, our results indicate that these interactions are increased in response to wild-type polyoma virus infection (FIG. 6).

To determine if Taz interacts with proteins other than T antigens, we used a purified anti-Taz polyclonal antibody cross-linked to Protein A in immunoprecipitation experiments. We used this antibody, as well as a normal IgG control antibody, to immunoprecipitate proteins from extracts of BMK, BNL, and P19 (embryonic carcinoma) cells. The immunoprecipitates were washed and analyzed by polyacrylamide gel electrophoresis followed by Coomassie Blue staining. The bands that differed between the anti-Taz and control lanes were cut out and subjected to standard mass spectrometry techniques. From the mass spectrometry results, we identified four additional proteins that interact with TAZ, ras-GTPase-activating protein SH3-domain binding protein (GAP SH3 binding protein or G3BP) (GenBank Accession Number 7305075), Nucleolin (GenBank Accession Number 13529464), Vesicle Associated Protein 1 (GenBank Accession Number 7514116), and Death Inducer with SAP domain (DIS).

G3BP is also known as human DNA helicase VIII. Like other proteins that are part of the Ras signal transduction pathway, G3BP is overexpressed in human tumors. In addition, this protein regulates S phase entry (Guitard et al., Cancer Lett. 162:213-221, 2001; Costa et al., Nucleic Acids Res. 27:817-821, 1999; and Tocque et al., Cell Signal 9:153-158, 1997).

Nucleolin, which is also known as human DNA helicase IV, is a multifunctional major nucleolar phosphoprotein. Nucleolin acts as an RNA binding protein, an autoantigen, a transcriptional repressor, and a switch-region targeting factor. In addition, nucleolin exhibits autodegradation, DNA and RNA helicase activities, and DNA-dependent ATPase activity (Srivastava et al., FASEB J. 13:1911-1922, 1999; Tuteja and Tuteja, Crit. Rev. Biochem. Mol. Biol. 33:407-436, 1998; and Ginisty et al., J. Cell Sci. 112:761-772, 1999).

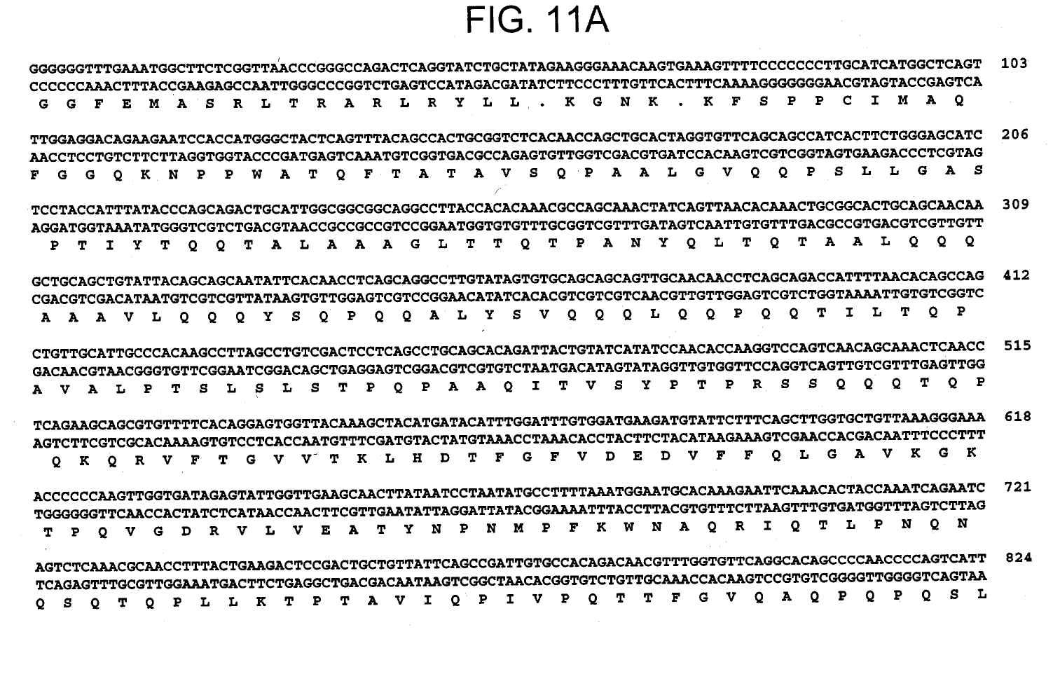

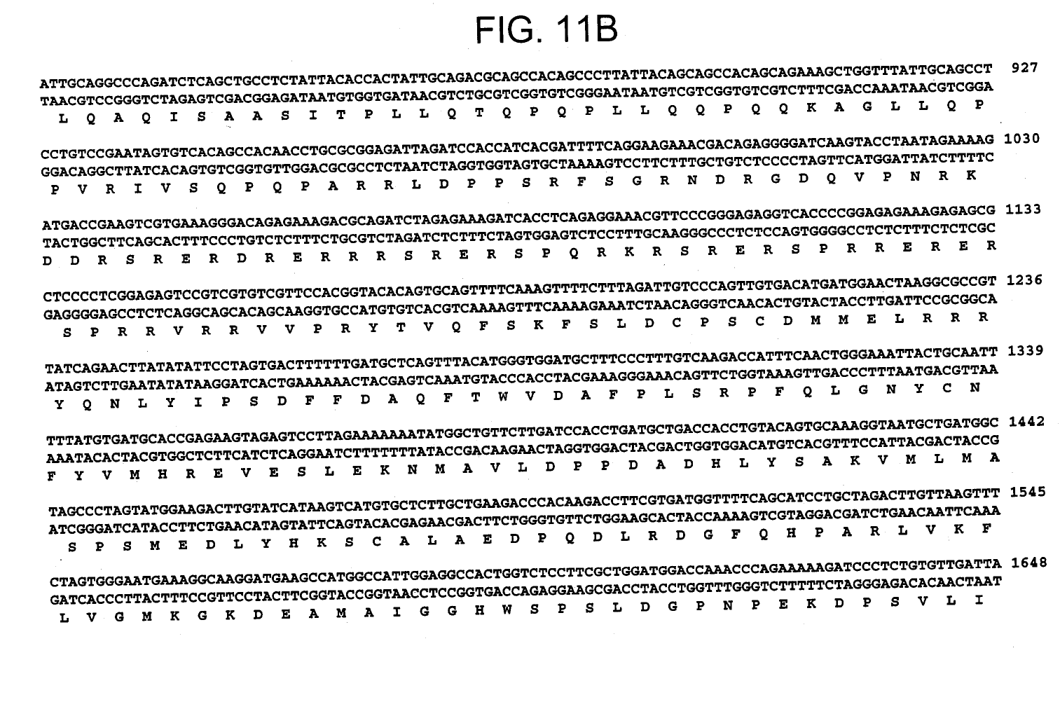

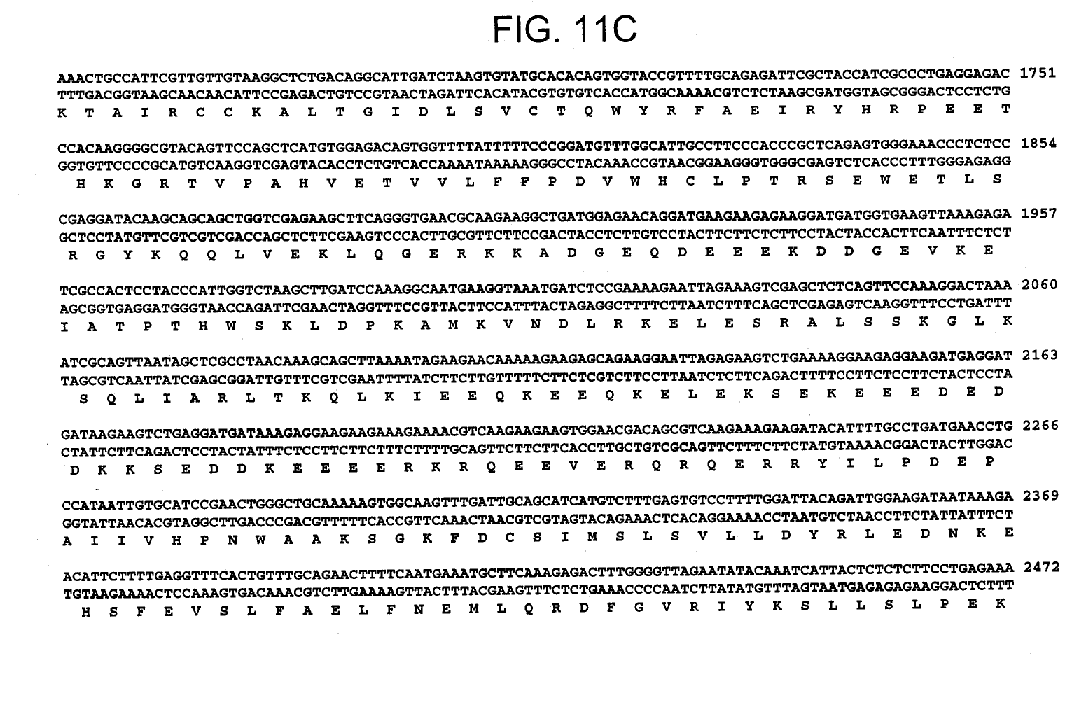

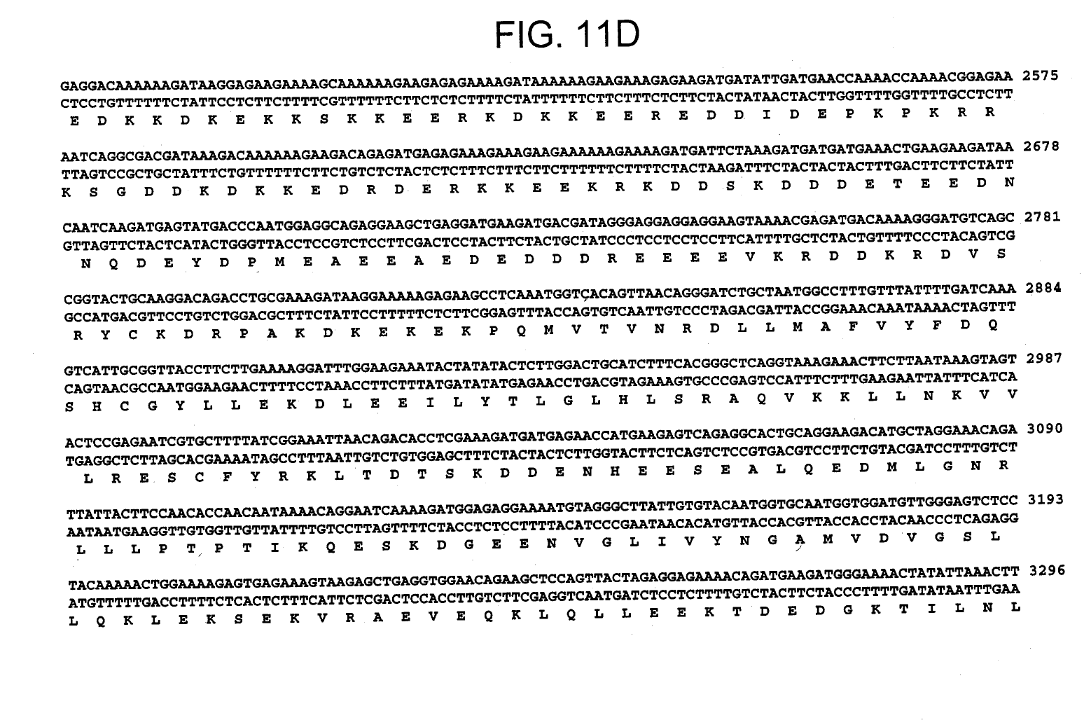

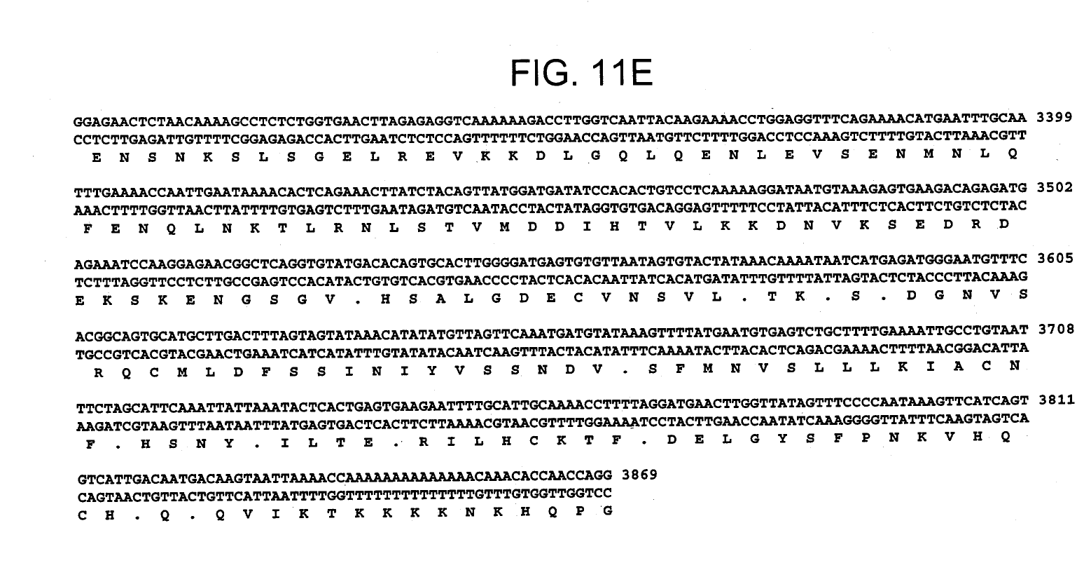

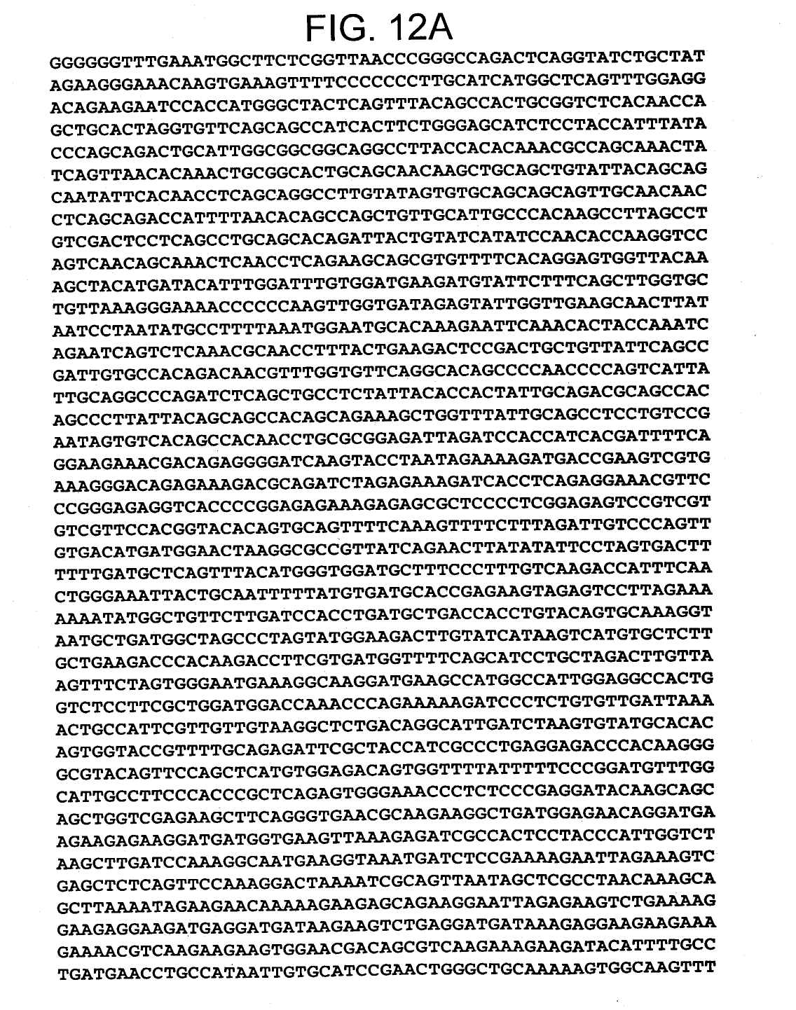

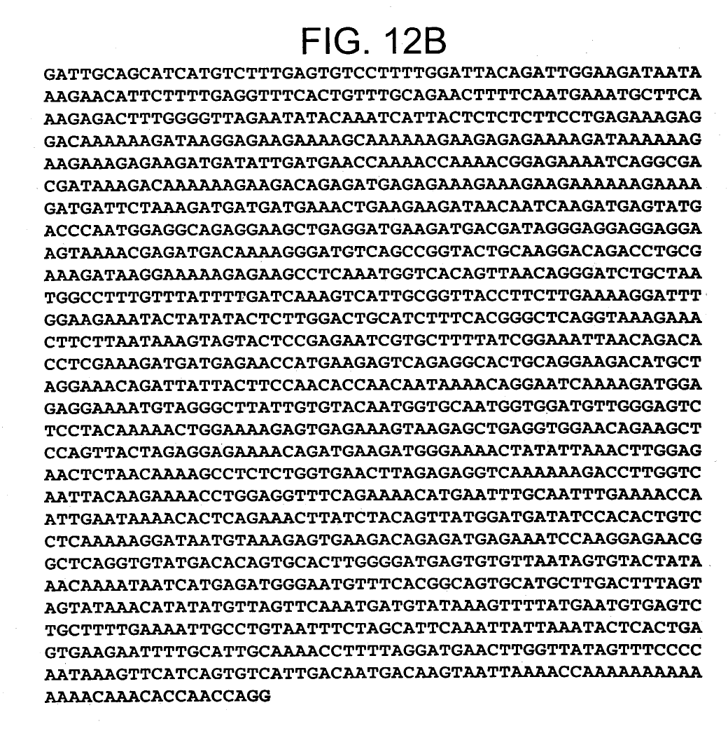

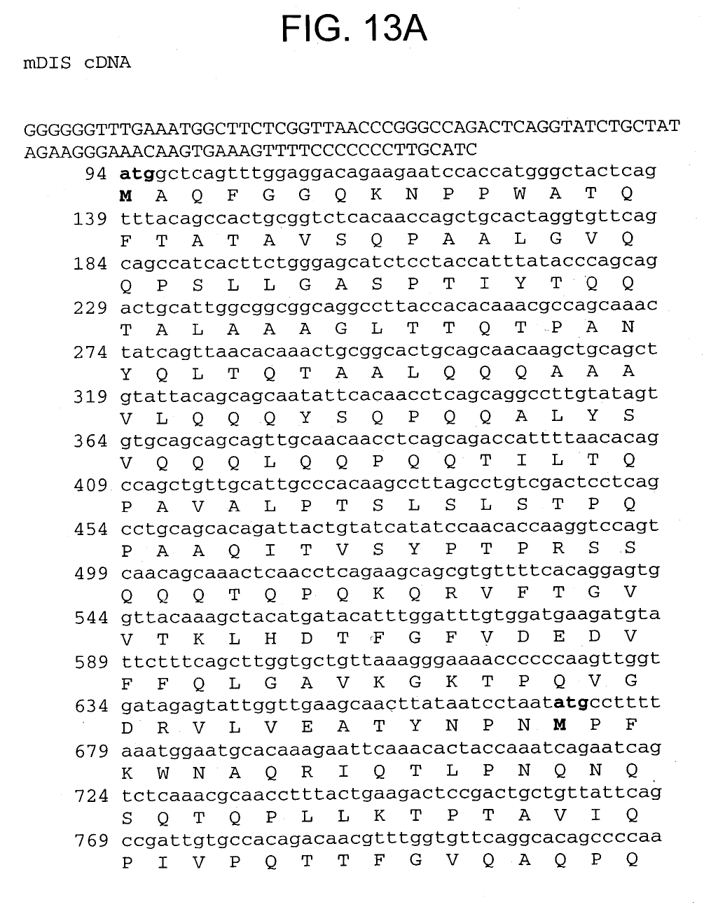

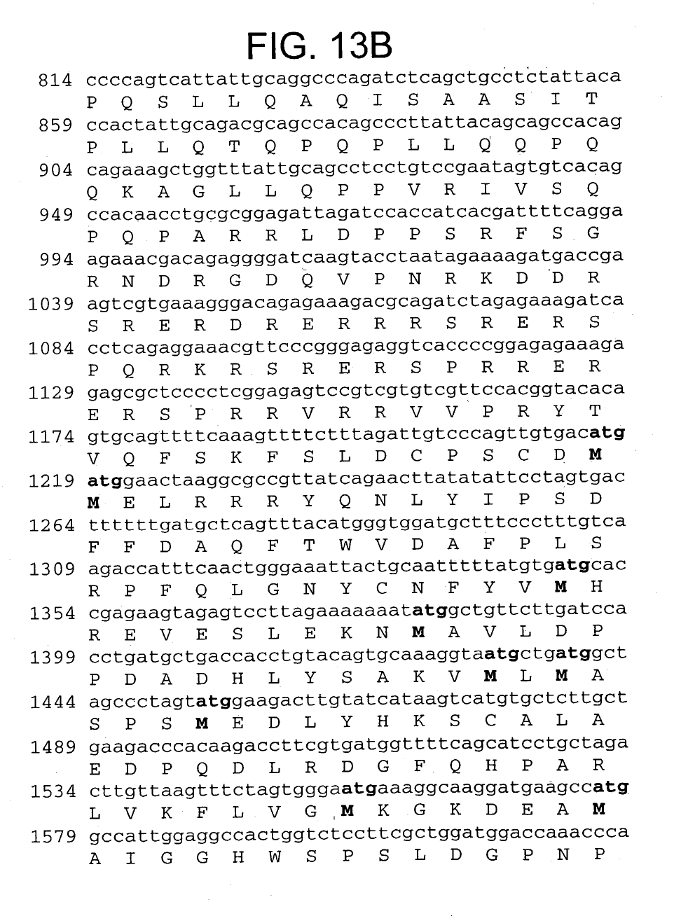

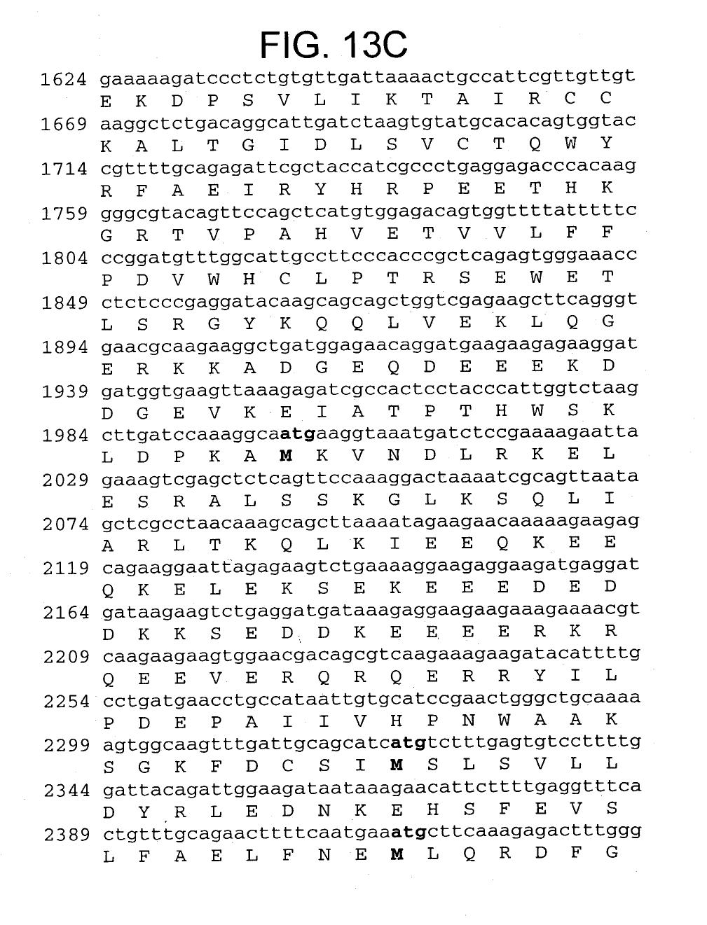

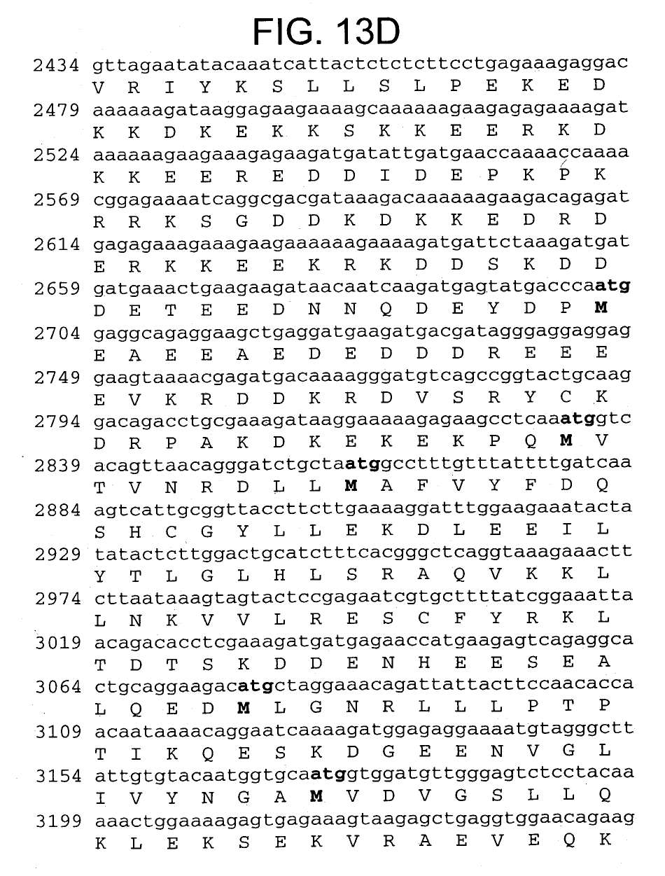

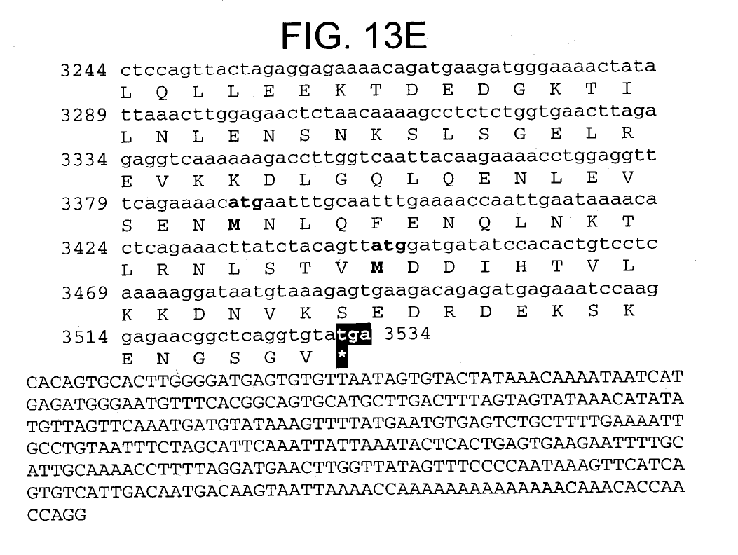

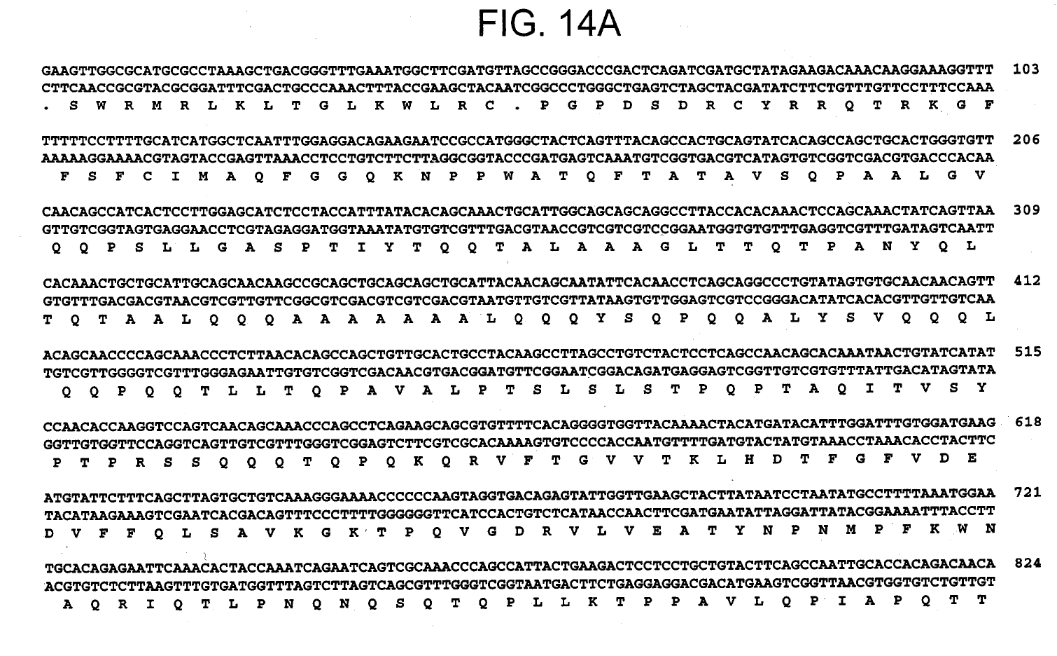

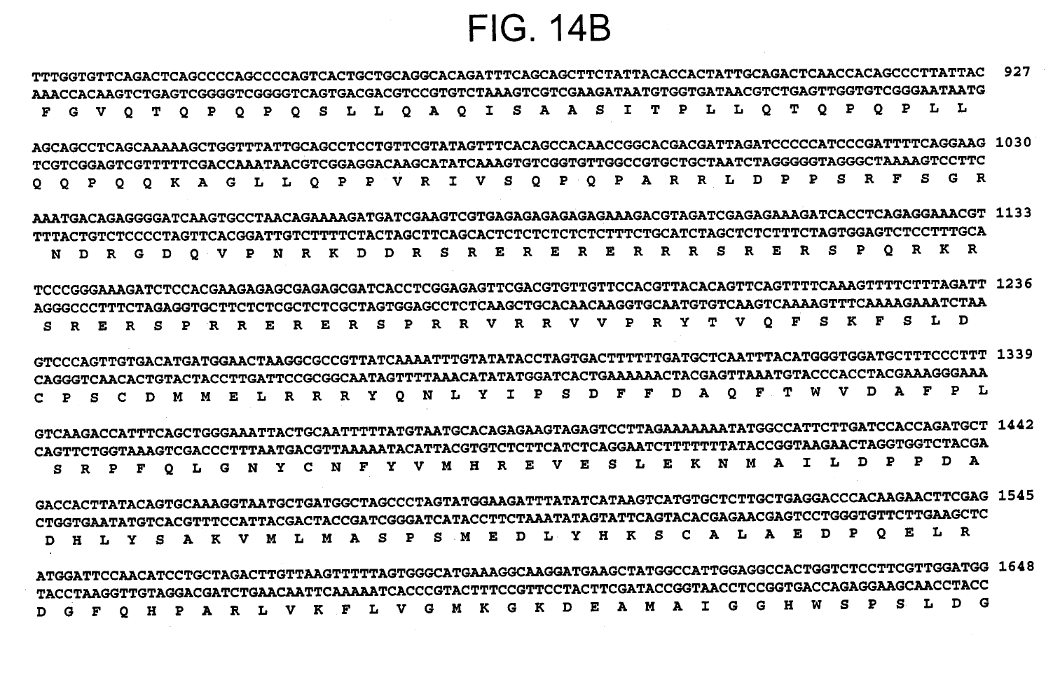

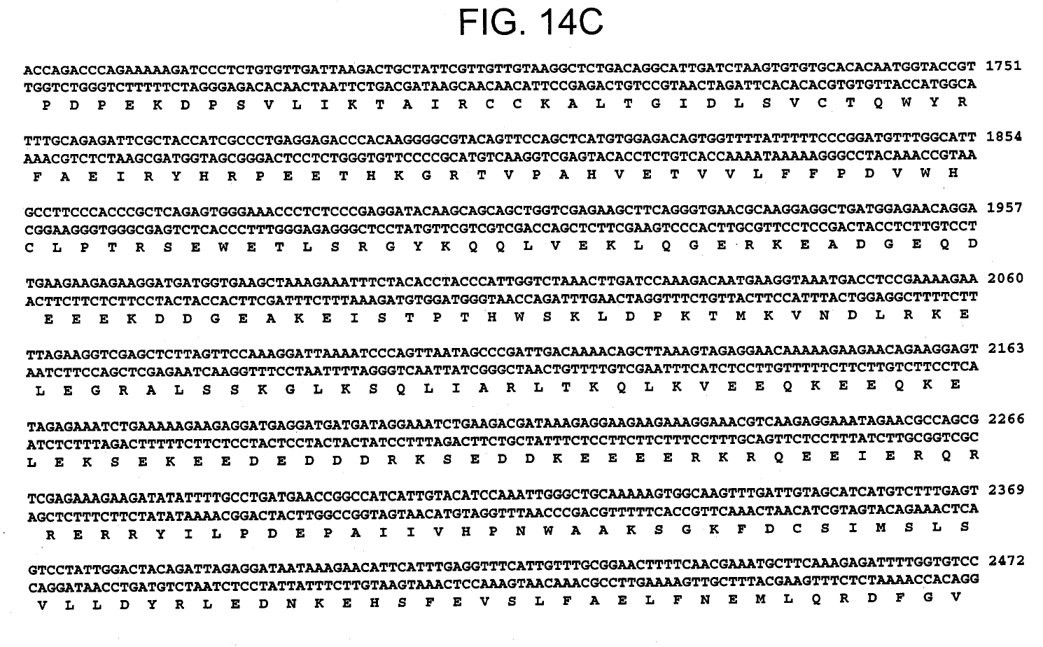

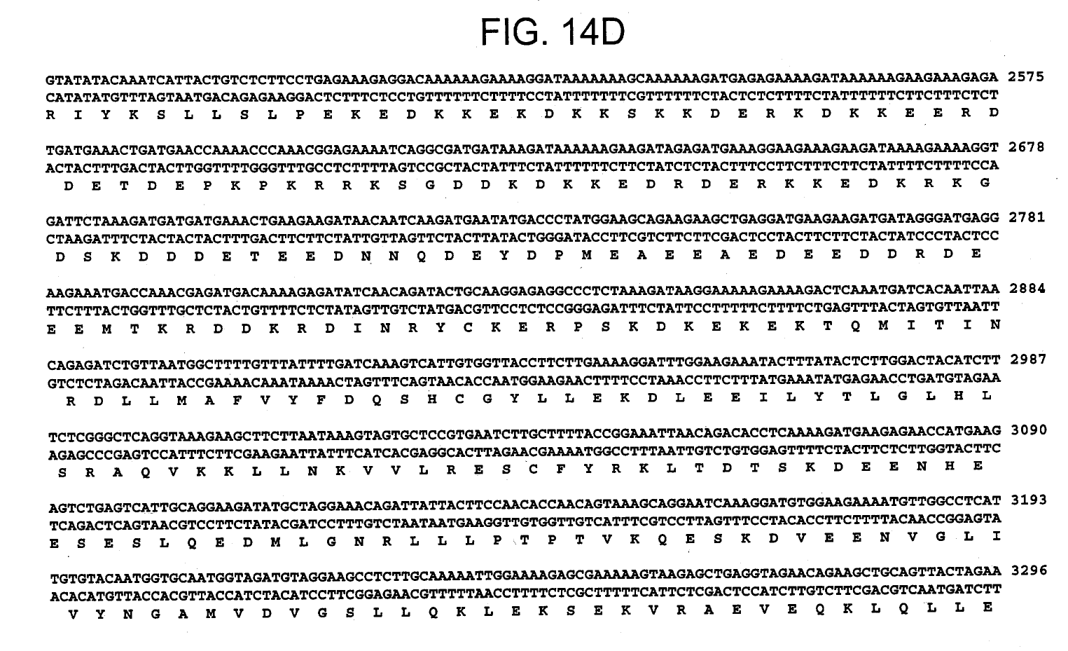

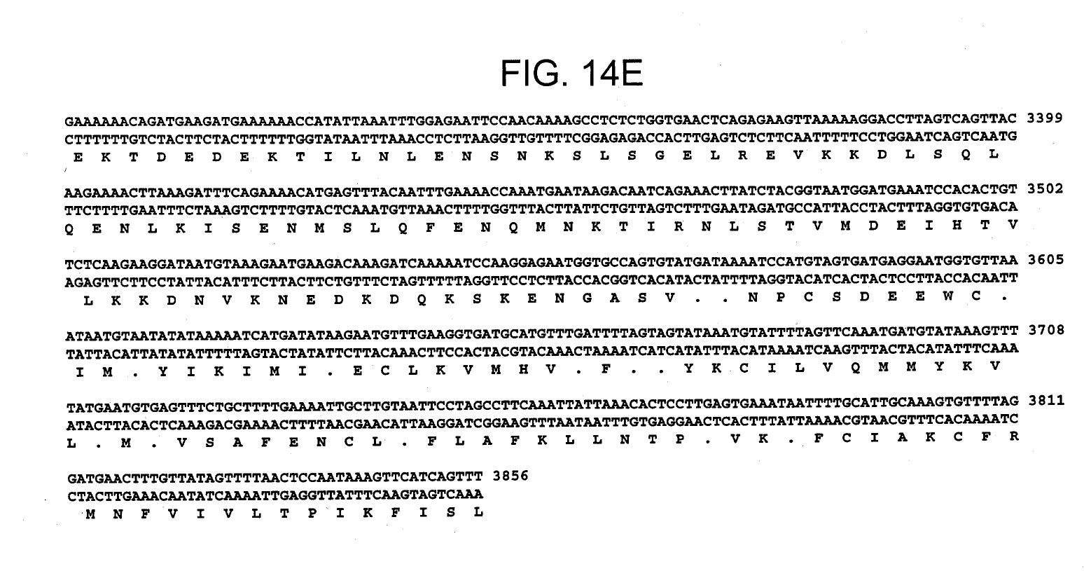

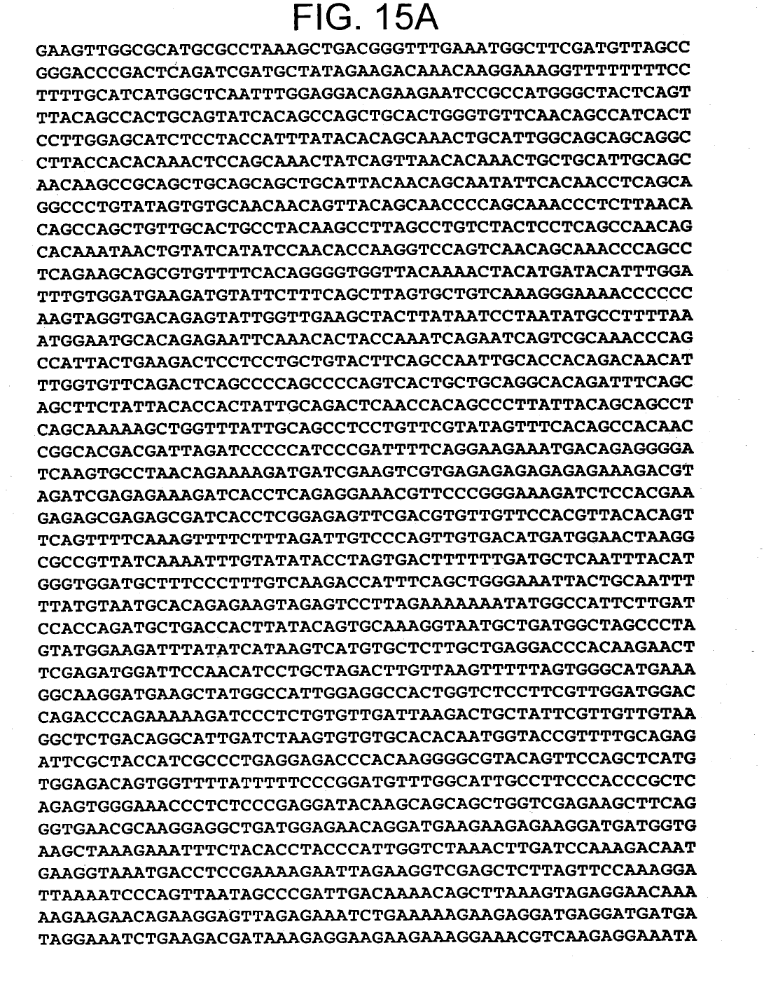

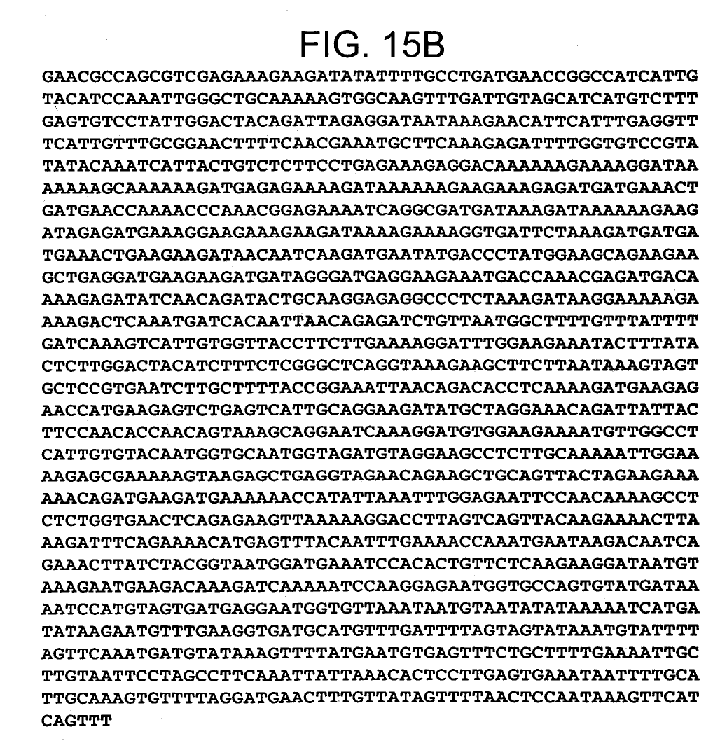

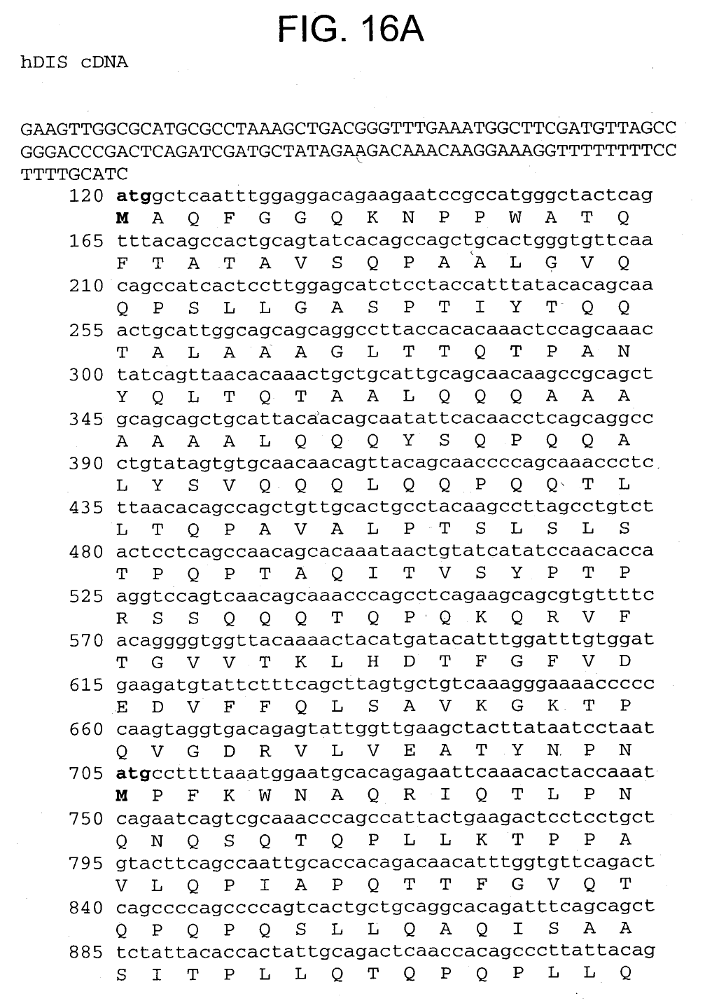

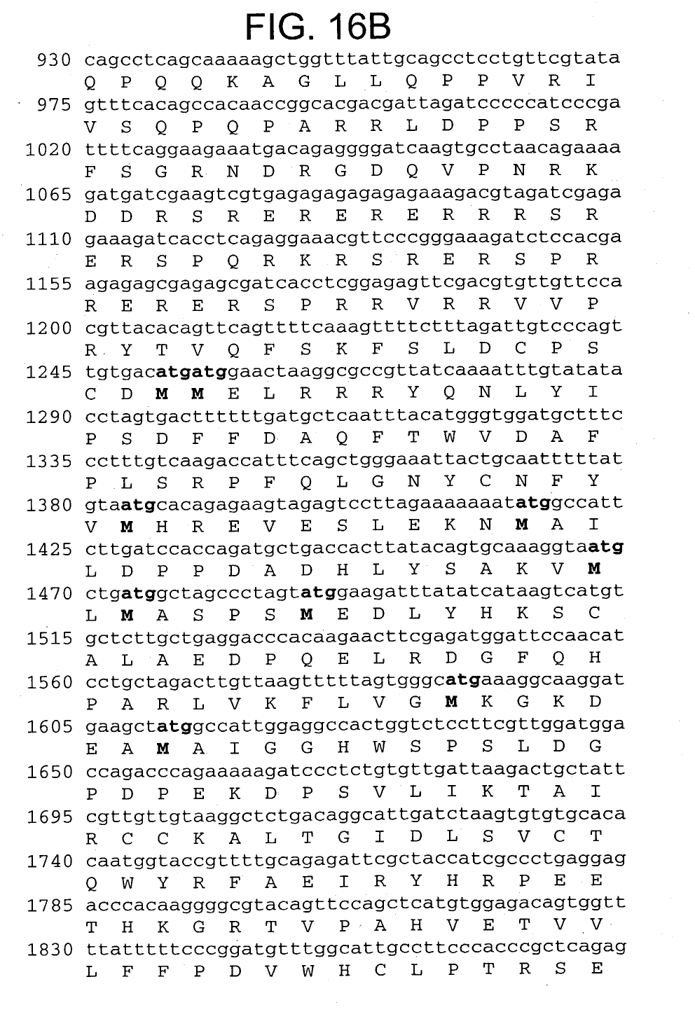

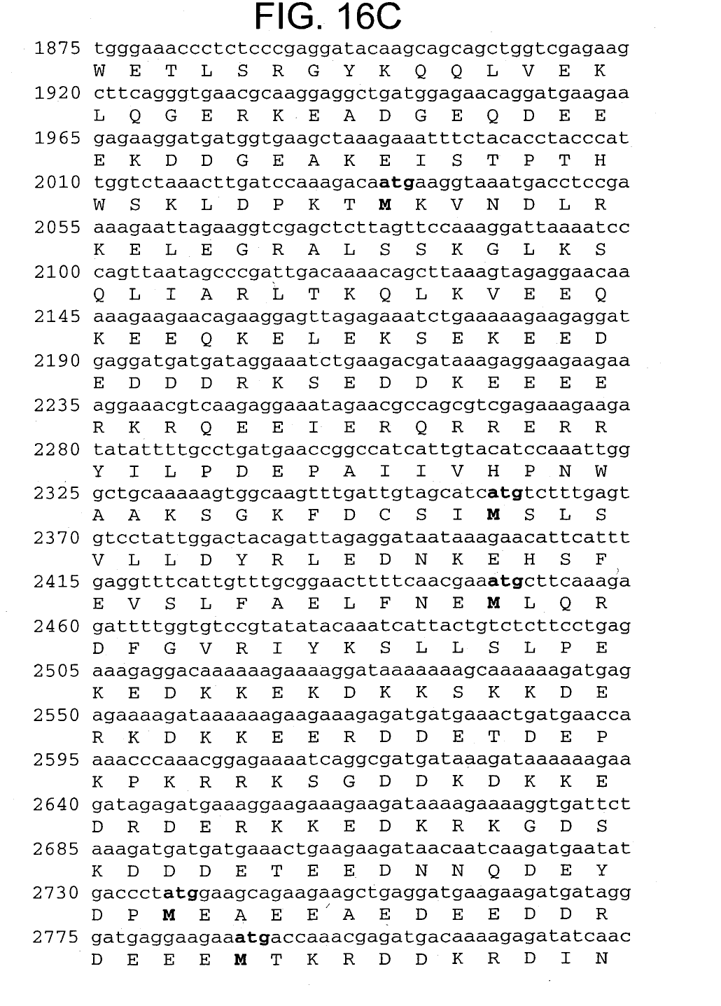

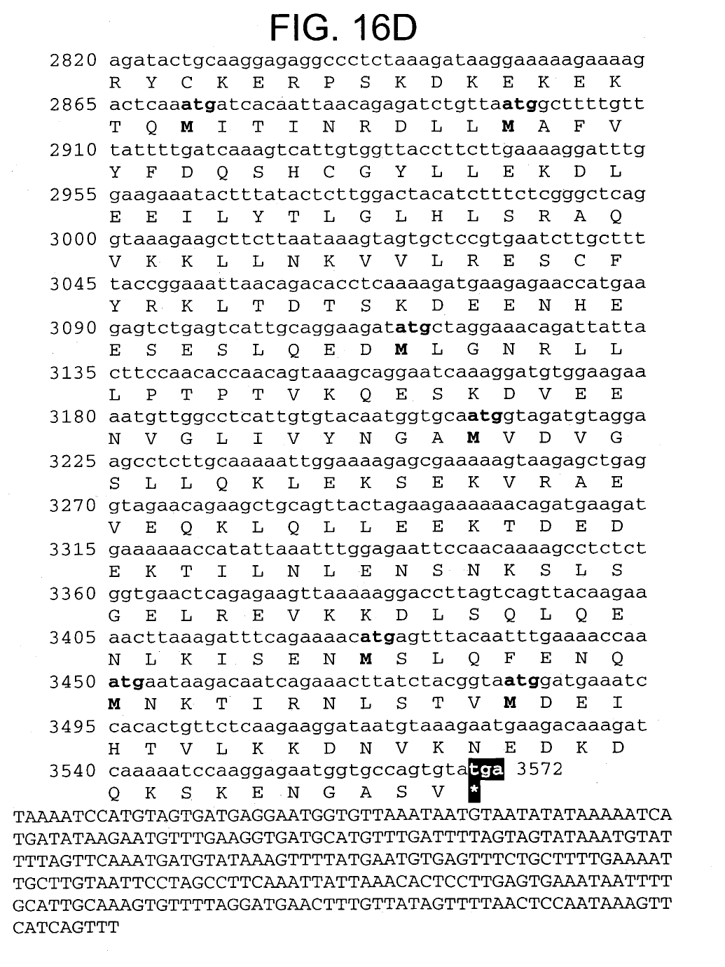

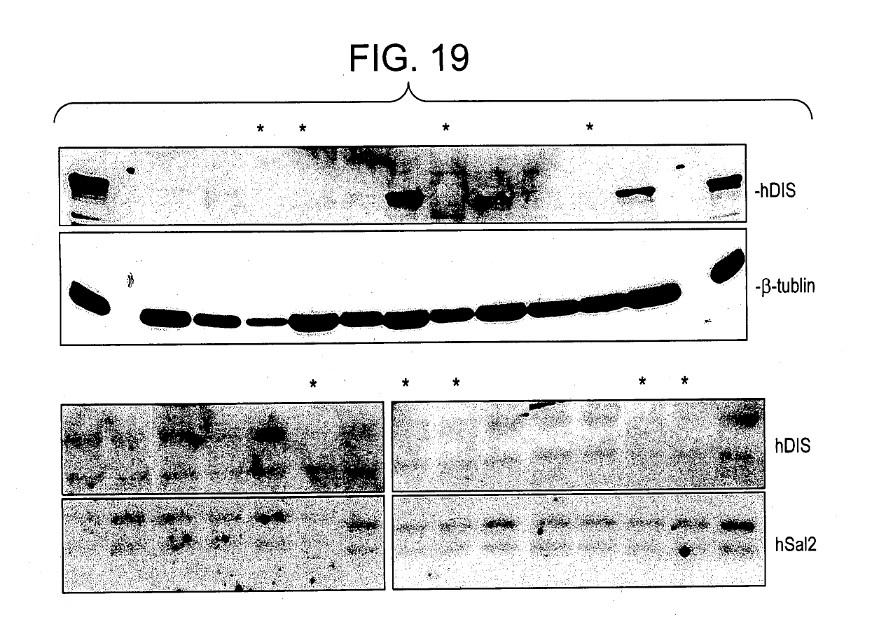

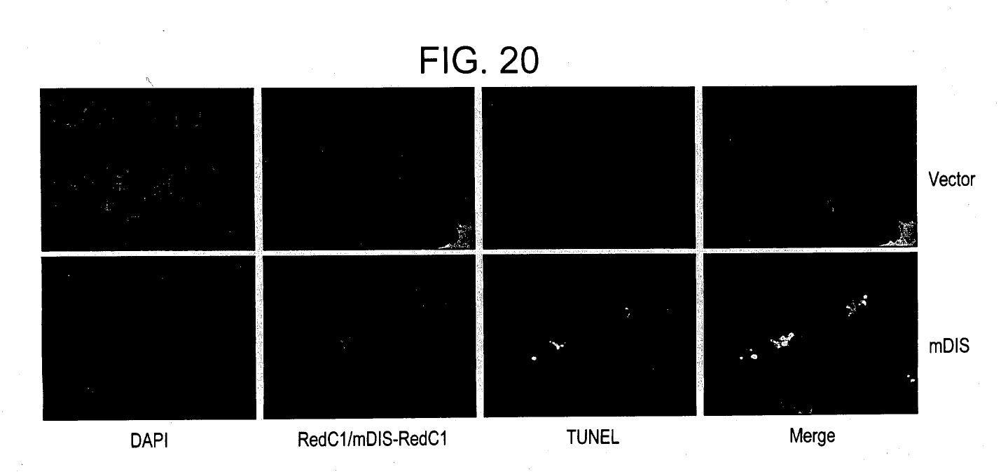

DIS, on the other hand, is a novel protein. We cloned both the mouse and the human cDNAs encoding this protein, the sequences of which are provided in FIGS. 11-16. DIS contains an SAP domain, which is a putative DNA-binding motif involved in chromosomal organization (Aravind et al., Trends Biochem. Sci. 25:112-114, 2000). The human DIS gene is located at chromosomal position 10Q22.1 where a loss of heterozygosity has been reported in endometrial and prostate adenocarcinomas. We show that a loss of heterozygosity for DIS exists in human ovarian tumors (FIG. 19, panel A) and that DIS is not expressed in ovarian tumors (FIG. 19, panel B). A loss of heterozygosity may be determined by standard methods in the art, for example, by using Southern blots, by sequencing, or by PCR analysis (See, for example, Debelenko et al., Hum. Mol. Genet. 6:2285-2290, 1997; and Emmert-Buck et al., Cancer Res. 55:2959-2962, 1995). In addition, we showed that DIS efficiently induces apoptosis in cultured cells. We transfected NIH 3T3 cells with DIS fused with Red (pREDC1, Clontech), and observed that the expression of DIS results in DNA condensation and in TUNEL positive cells (FIG. 20). These two markers indicate that cells are undergoing apoptosis.

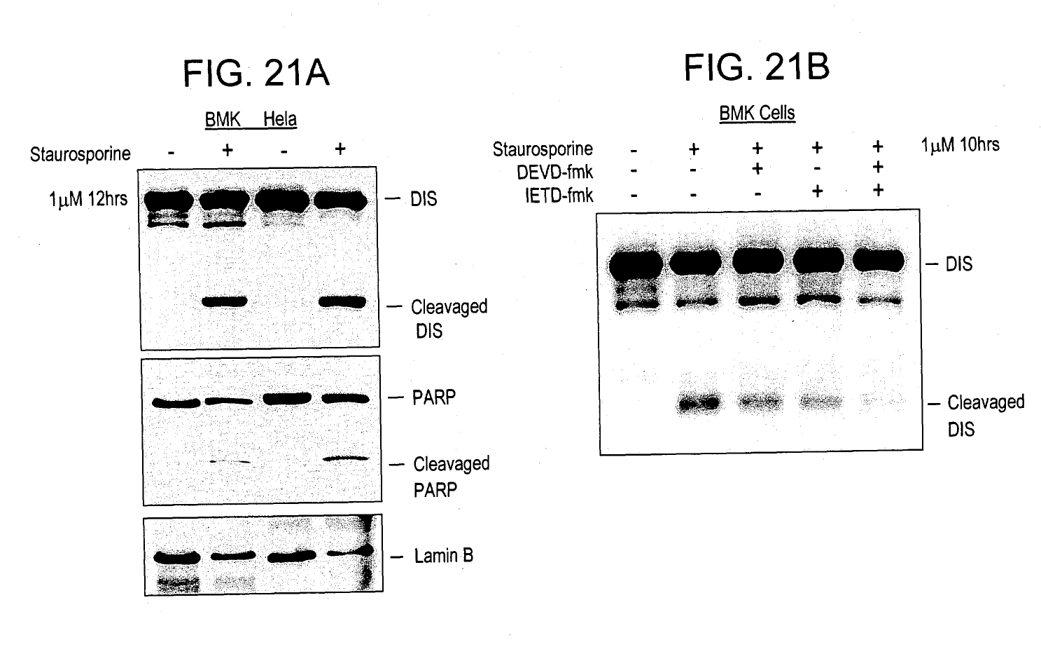

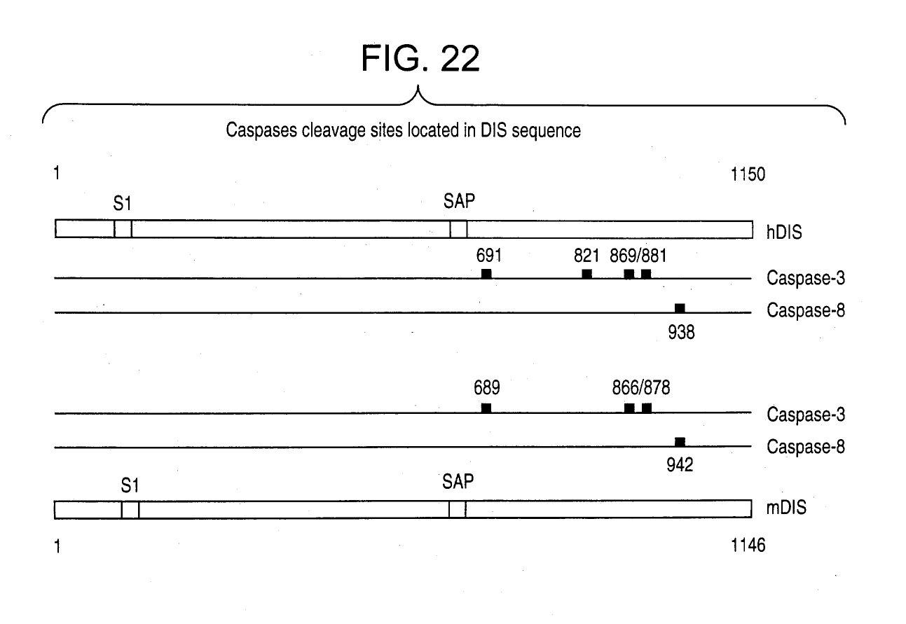

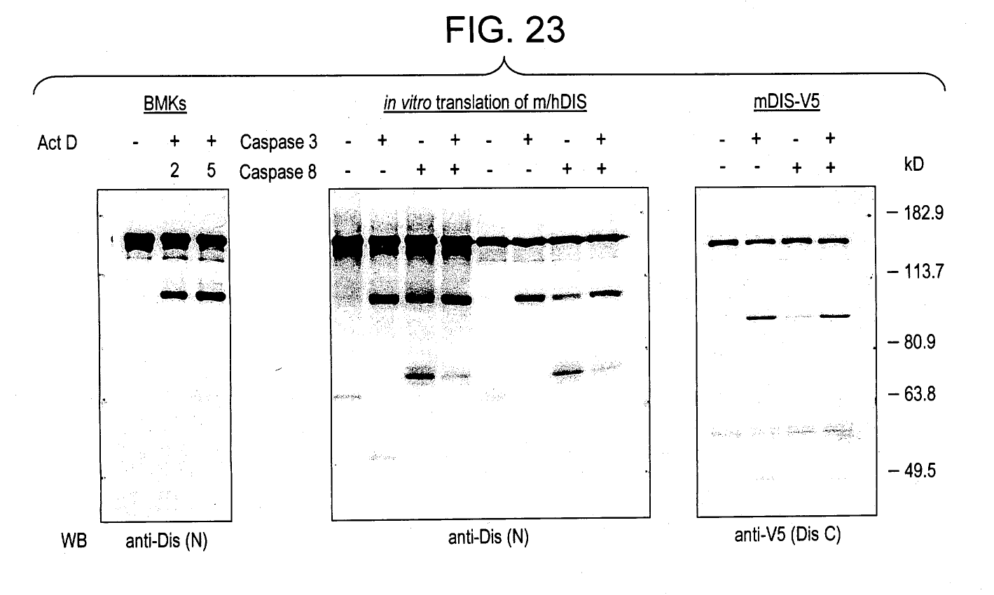

Furthermore, when we induced apoptosis in BMK and HeLa cells with staurosporine, we observed that DIS was degraded and that PARP and LaminB were cleaved (FIG. 21, panel A). Both PARP and LaminB are cleaved by caspases during apoptosis. We also observed that caspase-3 and caspase-8 inhibitors inhibited cleavage of DIS (FIG. 21, panel B). In view of these results, we analyzed the structure of human and mouse DIS and identified a number of caspase-3 and caspase-8 cleavage sites (FIG. 22). In vitro caspase cleavage experiments showed that DIS is sensitive to caspase-3 and that the first caspase-3 site (at amino acid 691 in human DIS (SEQ ID NO:4) and amino acid 689 in murine DIS (SEQ ID NO:2)) is used for cleavage (FIG. 23). Consequently, DIS is likely to function in regulating apoptosis and may be used in methods to diagnose and treat proliferative disorders.

Characterization of TAZ

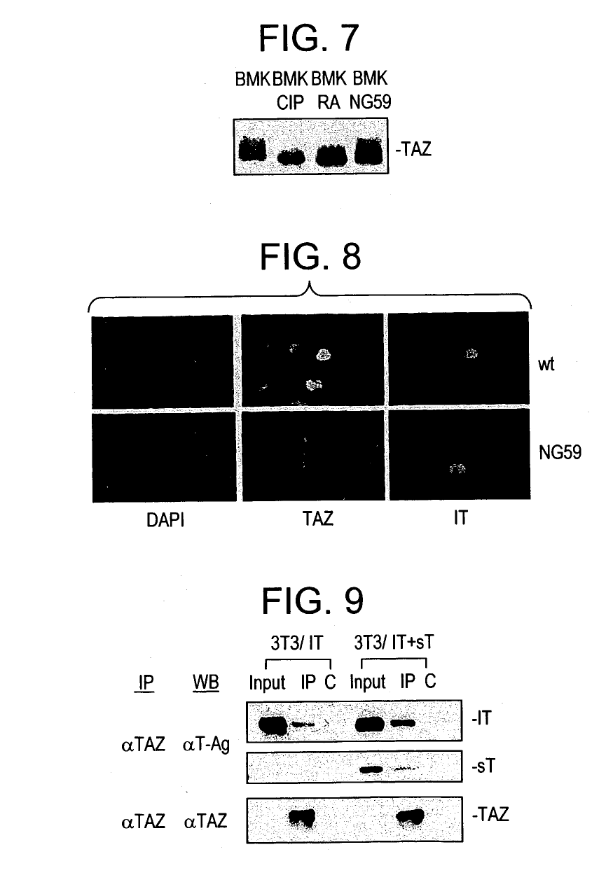

As is noted above, Taz also interacts with PP2A, a phosphatase. Accordingly, we looked at the phosphorylation state of murine Taz and discovered that murine Taz exists in a multiply phosphorylated state in uninfected BMK cells. Taz undergoes dephosphorylation when BMK cells are infected with wild-type polyoma virus, but not when BMK cells are infected with a mutant (NG 59) polyoma virus defective for the middle T antigen. As a control for these experiments, we determined the unphosphorylated state of Taz by adding calf intestinal phosphatase (CIP) to a BMK extract prior to Western blotting (FIG. 7).

Furthermore, we analyzed the intracellular localization of Taz in BMK cells and in these cells infected with either wild-type or NG 59 mutant polyoma virus. The nuclei of the cells were visualized by staining the DNA with DAPI and we used antibodies against Taz and the large T antigen to visualize these proteins. As is seen in FIG. 8, nuclear staining specific for Taz is enhanced in response to BMK cells being infected with wild-type polyoma virus, but not in response to an infection with NG 59 mutant polyoma virus. Additional immunoprecipitation experiments showed that the small T antigen increases binding of Taz to the large T antigen (FIG. 9). These results, in combination with those discussed above, indicate that nuclear import of Taz occurs after binding to the small T antigen and after undergoing dephosphorylation.

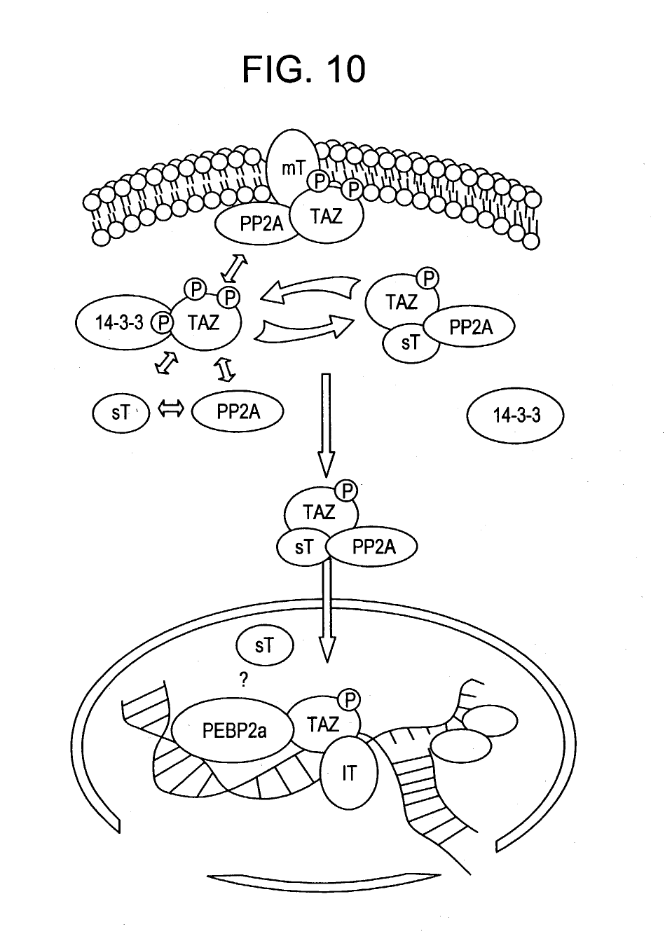

One model of how Taz may function during a polyoma virus infection is shown in FIG. 10. In this non-limiting example Taz may exist in both the nucleus and the cytoplasm. In the cytoplasm, phosphorylated Taz may bind to 14-3-3. After polyoma virus infection, Taz may interact with the small and middle T antigens. These T antigens, in turn, may recruit PP2A to the complex and Taz may undergo dephosphorylation and may dissociate from 14-3-3. Once dephosphorylated, Taz may enter the nucleus where it may bind to the large T antigen (and possibly PEBP2α), and may regulate replication and transcription.

Further support for a role for Taz in regulating transcription comes from data showing that Taz binds to the proline-rich regions of Runx1 (PEBP2α) transcription activator (Kanai et al., EMBO J. 19:6778-6791, 2000). The proline-rich domain that is involved in the interaction between Taz and Runx1 is also found in a number of other transcription factors, including c-Jun, AP-2, C/EBPα, NF-E2, KROX-20, KROX-24, Oct-4, MEF2B, and in the p53 homologue p73. In addition, alterations in members of the Runx family of transcription factors have been found in multiple leukemias and other human cancers (Lo Coco et al., Haematologica 82:364-370, 1997; and Glassman, Clin. Lab. Med. 20:39-48, 2000).

In addition, Taz is closely related to YAP (c-yes-associated protein), a cellular protein identified by its interaction with the SH3 domain of the c-yes proto-oncogene, a member of the Src family of protein tyrosine kinases (Sudol et al., J. Biol. Chem. 270:14733-14741, 1995). Since we show that Taz binds to c-src itself (FIG. 6), and since the interaction between the middle T antigen and c-src is known to play a central role in cell transformation and tumorigenesis by polyoma, Taz is also likely to function in oncogenic pathways.

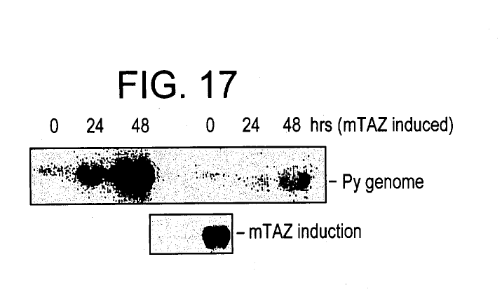

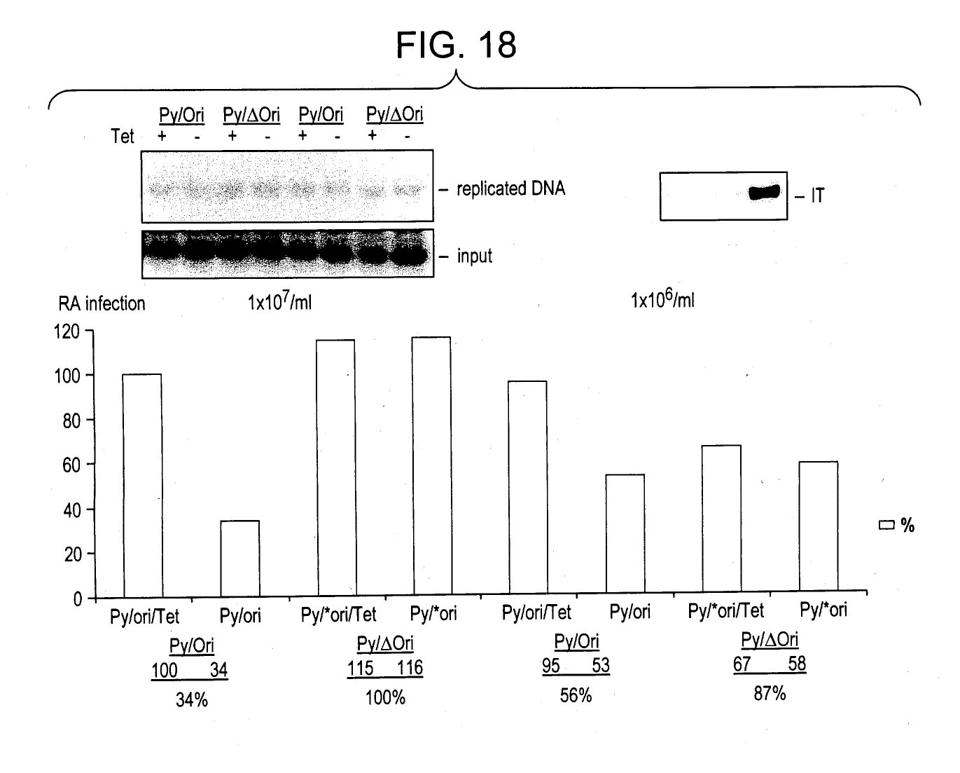

The importance of the interaction between Taz and polyoma T antigens is further demonstrated by our experiments where we showed that over-expression of Taz results in inhibition of polyoma virus replication. For these experiments, we used a 3T3 derived cell line that harbors a tetracycline-regulated mTaz gene. We induced mTaz expression by removing tetracycline 16 hours before infecting these cells, as well as control cells in which mTaz expression was not induced, with wild-type polyoma virus. After infection, we extracted low molecular weight DNA at different time points and performed a Southern blot using ³²P-labeled DNA corresponding to the polyoma origin of replication as a probe (FIG. 17). In addition, our polyoma DNA replication assay results show that TAZ that inhibition of origin replication driven by the virus depends on PEBP2-alpha binding sites located in the origin (FIGS. 18A and 18B). PEBP2-alpha is a member of the Runx transcription factor family. Modifications in Runx family members have been implicated in human leukemias and other cancers.

Based on the results of these experiment, we conclude that polyoma virus replication is strongly inhibited in cells over-expressing mTaz. Moreover, in combination with our other observations, these results indicate that an interaction between Taz and polyoma T antigens likely is necessary for a virus to replicate and disseminate and that providing additional Taz, more than can be bound by polyoma T antigens, results in a reduction in the ability of the virus to effectively propagate. Consequently, Taz nucleic acids and amino acids are likely to be desirable anti-viral agents.

In short, in view of the data presented above, including that Taz binds the proto-oncogene product c-src, that Taz binds to multiple transcription factors known to be associated with human cancers including those in the Runx family, e.g., PEBP2α, and that the BMD-13 T-HR mutant, which fails to interact with Taz, also fails to transform cells, Taz is likely to be linked to oncogenic pathways. Accordingly, Taz nucleic and amino acids may be used in a variety of methods to diagnose and treat proliferative disorders.

Treatment

The invention also provides a method of killing an abnormally proliferating cell using a BMD-13 T-HR mutant virus. Such T-HR mutants can be used to specifically target and destroy cancer cells in an organism. Since these mutant viruses can only propagate in cells that carry a mutation in a cellular gene that the virus would normally have to activate, in the case of proto-oncogene, or inactivate, in the case of a tumor suppressor gene, in order to replicate and disseminate, propagation of such a mutant virus would be specific to abnormal cells. Therefore, T-HR mutants can be used to specifically eliminate cancer cells from a patient. For example, a T-HR mutant (e.g., a polyoma virus carrying an alteration in any T antigen causing it to be defective in replication and tumor induction) may be used to selectively kill human leukemia cells that carry a genetic lesion in a Taz gene.

The therapeutic BMD-13 T-HR mutant may be administered by any of a variety of routes known to those skilled in the art, such as, for example, intraperitoneal, subcutaneous, parenteral, intravenous, intramuscular, or subdermal injection. However, the T-HR mutant may also be administered as an aerosol, as well as orally, nasally, or topically. Standard concentrations used to administer a BMD-13 T-HR mutant include, for example, 10², 10³, 10⁴, 10⁵, or 10⁶ plaque forming units (pfu)/animal, in a pharmacologically acceptable carrier. Appropriate carriers or diluents, as well as what is essential for the preparation of a pharmaceutical composition are described, e.g., in Remington's Pharmaceutical Sciences (18^{th} edition), ed. A. Gennaro, 1990, Mack Publishing Company, Easton, Pa., a standard reference book in this field.

Formulations for parenteral administration may, for example, contain excipients, sterile water, or saline. For inhalation, formulations may contain excipients, for example, lactose. Aqueous solutions may be used for administration in the form of nasal drops, or as a gel for topical administration. The exact dosage used will depend on the severity of the condition (e.g., the size of the tumor), or the general health of the patient and the route of administration. The T-HR mutant may be administered once, or it may be repeatedly administered as part of a regular treatment regimen over a period of time.

In addition, the invention provides methods of killing an abnormally proliferating cell using a target gene of a tumor virus. Such target genes, for example, Taz, may be identified using a T-HR according to the methods of the invention. A Taz nucleic acid sequence, or a nucleic acid sequence encoding a protein that interacts with Taz, e.g., a DIS nucleic acid sequence, may be introduced into an abnormally proliferating cell, for example, by using liposome-based transfection techniques, to treat the proliferative disorder (Units 9.1-9.4, Ausubel et al., Current Protocols in Molecular Biology, John Wiley & Sons, New York (1995)). Such DNA constructs may also be introduced into mammalian cells using an adenovirus, or retroviral or vaccinia viral vectors (Units 9.10 and 16.15-16.19, Ausubel et al., Current Protocols in Molecular Biology, John Wiley & Sons, New York (1995)). These standard methods of introducing DNA into cells are applicable to a variety of cell-types.

For example, recombinant adenoviral vectors offer several significant advantages for gene transfer. The viruses can be prepared at extremely high titer, infect non-replicating cells, and confer high-efficiency and high-level transduction of target cells in vivo after directed injection or perfusion. Either directed injection or perfusion would be appropriate for delivery of vectors containing a T-HR target gene in a clinical setting. Moreover, transient expression may be sufficient to remove the abnormally proliferating cells and it may be desirable in view of possible bio-safety or toxicity concerns associated with long-term expression of a T-HR target gene.

In animal models, adenoviral gene transfer has generally been found to mediate high-level expression for approximately one week. The duration of transgene expression may be prolonged, and ectopic expression reduced, by using tissue-specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a Cre-Lox strategy (Engelhardt, et al., Proc. Natl. Acad. Sci. USA 91:6196-6200, 1994; Kochanek, et al., Proc. Natl. Acad. Sci. USA 93:5731-5736, 1996).

In addition, recombinant adeno-associated viruses (rAAV), derived from non-pathogenic parvoviruses, may be used to express a T-HR target gene as these vectors evoke almost no cellular immune response, and produce transgene expression lasting months in most systems. Incorporation of a tissue-specific promoter is, again, beneficial. Furthermore, besides adenovirus vectors and rAAVs, other vectors and techniques are known in the art, for example, those described by Wattanapitayakul and Bauer (Biomed. Pharmacother. 54:487-504, 2000), and citations therein.

A vector carrying a T-HR target gene can be delivered to the target organ through in vivo perfusion by injecting the vector into the target organ, or into blood vessels supplying this organ (e.g., for the liver, the portal vein (Tada, et al., Liver Transpl. Surg. 4:78-88, 1998) could be used, or in the case of leukemia, the blood itself may be the delivery target.

Furthermore, a target gene of a T-HR mutant, for example, Taz, GAP SH3 binding protein, nucleolin, Vesicle Associated Protein 1, or DIS, may also be used as an anti-viral agent. In the case of tumor suppressor genes, a DNA tumor virus generally needs to inactivate the gene to replicate and disseminate. Accordingly, providing active forms of such genes to a cell, or over-expressing these genes in the cell, would effectively interfere with virus replication and dissemination, and, thereby, reduce the ability of the virus to cause or contribute to a proliferative disorder. Alternatively, an anti-sense nucleic acid for a proto-oncogene may be used to inactivate such a gene in a cell and, thereby, prevent a DNA tumor virus from activating the gene, or in the case of an abnormally proliferating cell, to decrease or halt abnormal proliferation.

Test Compounds

Compounds that may be tested for the ability to modulate the expression of target genes of T-HR mutants, or of their gene products, can be from natural as well as synthetic sources. Those skilled in the field or drug discovery and development will understand that the precise source of test extracts or compounds is not critical to the methods of the invention. Examples of such extracts or compounds include, but are not limited to, plant-based, fungal-based, prokaryotic-based, or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

A test compound that modulates the expression of a T-HR target gene, or its encoded protein, may be used to treat proliferative diseases such as leukemias and other types of cancer.

Diagnosis

The methods of the present invention can be used to diagnose abnormally proliferating cells in a patient by determining whether the cells of the patient can act as permissive hosts for the growth of a BMD-13 T-HR mutant virus. As described above, a permissive host for the growth of this mutant virus has a mutation in a cellular gene, e.g., a Taz, GAP SH3 binding protein, nucleolin, Vesicle Associated Protein 1, or DIS gene, that is the target for the wild-type viral protein that corresponds to the mutant viral protein. This cellular mutation is believed to compensate for the modification of function in a particular gene in the T-HR mutant and contribute to the abnormal phenotype of the cell. However, the permissive host may also have a mutation in a cellular gene encoding a protein that interacts with a protein that binds to a viral protein, or in a cellular gene that encodes a component of a signaling pathway that is required for viral transformation. This information then may be used to screen a population as a whole for individuals that are at an increased risk of developing a particular type of proliferative disorder and also may be used to further characterize the cancer cell (e.g., to grade the stage to which the cancer has progressed).

For example, a BMD-13 T-HR mutant may be used to determine whether there is a genetic lesion in a Taz, G3BP, nucleolin, Vesicle Associated Protein 1, or DIS gene. Once identified, probes and primers based on this genetic lesion may be used as markers to detect the particular change in samples from other patients.

A genetic lesion in a candidate gene may be identified in a biological sample obtained from a patient using a variety of methods available to those skilled in the art. Generally, these techniques involve PCR amplification of nucleic acid from the patient sample, followed by identification of the genetic lesion by either altered hybridization, aberrant electrophoretic gel migration, restriction fragment length polymorphism (RFLP) analysis, binding or cleavage mediated by mismatch binding proteins, or direct nucleic acid sequencing. Any of these techniques may be used to facilitate detection of a genetic lesion in a candidate gene, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236 (1989)). Furthermore, expression of the candidate gene in a biological sample (e.g., a biopsy) may be monitored by standard Northern blot analysis or may be aided by PCR (see, e.g., Ausubel et al., Current Protocols in Molecular Biology, John Wiley & Sons, New York, N.Y. (1995); PCR Technology: Principles and Applications for DNA Amplification, H. A. Ehrlich, Ed., Stockton Press, New York; Yap et al., Nucl. Acids. Res. 19:4294 (1991)).

Once a genetic lesion is identified using the methods of the invention (as is described above), the genetic lesion is analyzed for association with an increased risk of developing a proliferative disorder.

Furthermore, antibodies against a protein produced by the gene included in the genetic lesion, for example the Taz, G3BP, Nucleolin, Vesicle Associated Protein 1, or DIS protein, may be used to detect altered expression levels of the protein, including a lack of expression, or a change in its mobility on a gel, indicating a change in structure or size. In addition, antibodies may be used for detecting an alteration in the expression pattern or the sub-cellular localization of the protein. Such antibodies include ones that recognize both the wild-type and mutant protein, as well as ones that are specific for either the wild-type or an altered form of the protein. We showed that a polyclonal rabbit anti-Taz antibody specifically recognizes Taz on Western blots and in cell culture. If desired, monoclonal antibodies may also be prepared using the Taz proteins described above and standard hybridoma technology (see, e.g., Kohler et al., Nature 256:495 (1975); Kohler et al., Eur. J. Immunol. 6:511 (1976); Kohler et al., Eur. J. Immunol. 6:292 (1976); Hammerling et al., In Monoclonal Antibodies and T Cell Hybridomas, Elsevier, New York, N.Y. (1981); Ausubel et al., Current Protocols in Molecular Biology, John Wiley & Sons, New York, N.Y. (2000)). Once produced, monoclonal antibodies are also tested for specific Taz protein recognition by Western blot or immunoprecipitation analysis (by the methods described in, for example, Ausubel et al. (Current Protocols in Molecular Biology, John Wiley & Sons, New York, N.Y. (2000)).

Antibodies used in the methods of the invention may be produced using amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf (CABIOS 4:181 (1988)). These fragments can be generated by standard techniques, e.g., by the PCR, and cloned into the pGEX expression vector (Ausubel et al., Current Protocols in Molecular Biology, John Wiley & Sons, New York, N.Y. (1995)). GST fusion proteins are expressed in E. coli and purified using a glutathione agarose affinity matrix as described in Ausubel et al. (Current Protocols in Molecular Biology, John Wiley & Sons, New York, N.Y., (1995)).

Use of Transgenic and Knockout Animals in Diagnosis

The disclosed transgenic and knock out animals may be used as research tools to determine genetic and physiological features of a cancer, and for identifying compounds that can affect leukemia and other cancers. Knockout animals also include animals where the normal gene has been inactivated or removed and replaced with a known polymorphic or other mutant allele of this gene. These animals can serve as a model system for the risk of acquiring a proliferative disease that is associated with a particular allele.

In general, the method of identifying markers associated with a proliferative disorder, such as leukemia, involves comparing the presence, absence, or level of expression of genes, either at the RNA level or at the protein level, in tissue from a transgenic or knock out animal and in tissue from a matching non-transgenic or knock out animal. Standard techniques for detecting RNA expression, e.g., by Northern blotting, or protein expression, e.g., by Western blotting, are well known in the art. Differences between animals such as the presence, absence, or level of expression of a gene indicate that the expression of the gene is a marker associated with a proliferative disorder, such as leukemia. The molecular markers, once identified, can be used to predict whether patients with a specific cancer will have indolent or aggressive disease, and may be mediators of disease progression. Identification of such mediators would be useful since they are possible therapeutic targets. Identification of markers can take several forms.

One method by which molecular markers may be identified is by use of directed screens. Patterns of accumulation of a variety of molecules that may regulate growth can be surveyed using immunohistochemical methods. Screens directed at analyzing expression of specific genes or groups of molecules implicated in pathogenesis can be continued during the life of the transgenic or knockout animal. Expression can be monitored by immunohistochemistry as well as by protein and RNA blotting techniques. Mestastatic foci, once formed, can also be subjected to such comparative surveys.

Alternatively, molecular markers may be identified using genomic screens. For example, tissue can be recovered from young transgenic or knockout animals (e.g, that may have early stage cancer) and older transgenic or knockout animals (e.g., that may have advanced stage cancer), and compared with similar material recovered from age-matched normal littermate controls to catalog genes that are induced or repressed as disease is initiated, and as disease progresses to its final stages. These surveys will generally include cellular populations present in the affected tissue.

This analysis can also be extended to include an assessment of the effects of various treatment paradigms (including the use of compounds identified as affecting cancers in the transgenic or knockout animals) on differential gene expression (DGE). The information derived from the surveys of DGE can ultimately be correlated with disease initiation and progression in the transgenic or knockout animals.

To assess the effectiveness of a treatment paradigm, a transgene, such as a mutant Taz gene, may be conditionally expressed (e.g., in a tetracycline sensitive manner). For example, the promoter for the Taz gene may contain a sequence that is regulated by tetracycline and expression of the Taz gene product ceases when tetracycline is administered to the mouse. In this example, a tetracycline-binding operator, tetO, is regulated by the addition of tetracycline, or an analog thereof, to the organism's water or diet. The tetO may be operably-linked to a coding region, for example a mutant Taz gene. The system also may include a tetracycline transactivator (tTA), which contains a DNA binding domain that is capable of binding the tetO as well as a polypeptide capable of repressing transcription from the tetO (e.g., the tetracycline repressor (tetR)), and may be further coupled to a transcriptional activation domain (e.g., VP16). When the tTA binds to the tetO sequences, in the absence of tetracycline, transcription of the target gene is activated. However, binding of tetracycline to the tTA prevents activation. Thus, a gene operably-linked to a tetO is expressed in the absence of tetracycline and is repressed in its presence. Alternatively, this system could be modified such that a gene is expressed in the presence of tetracycline and repressed in its absence. Tetracycline regulatable systems are well known to those skilled in the art and are described in, for example, WO 94/29442, WO 96/40892, WO 96/01313, and Yamamoto et al. (Cell 101:57-66 (2000).

In addition, the knockout organism may be a conditional, i.e., somatic knockout. For example, FRT sequences may be introduced into the organism so that they flank the gene of interest. Transient or continuous expression of the FLP protein may then be used to induce site-directed recombination, resulting in the excision of the gene of interest. The use of the FLP/FRT system is well established in the art and is described in, for example, U.S. Pat. No. 5,527,695, and in Lyznik et al. (Nucleic Acid Research 24:3784-3789 (1996)).

Conditional, i.e., somatic knockout organisms may also be produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a Taz gene, flanked by loxP sites is excised, generating a knockout. This system is described, for example, in Kilby et al. (Trends in Genetics 9:413-421 (1993)).

Particularly desirable is a mouse model for leukemia wherein the nucleic acid having an alteration in a Taz, GAP SH3 binding protein, nucleolin, Vesicle Associated Protein 1, or DIS gene, for example, an altered human Taz gene, is expressed in the blood cells of the transgenic mouse such that the transgenic mouse develops leukemia. The mice may also contain a T antigen transgene, such as one expressing an appropriate (e.g., N-terminally truncated) fragment of a T antigen under the control of a tissue specific promoter, or have a knockout of the murine Taz, GAP SH3 binding protein, nucleolin, Vesicle Associated Protein 1, or DIS gene. In addition, cell lines from these mice may be established by methods standard in the art.

Construction of transgenes can be accomplished using any suitable genetic engineering technique, such as those described in Sambrook et al. (Molecular Cloning: A Laboratory Manual, Cold Spring Harbor Laboratory, N.Y., (1989)). Many techniques of transgene construction and of expression constructs for transfection or transformation in general are known and may be used for the disclosed constructs. Although the use of an altered hTaz gene in the transgene constructs is used as an example, a wild-type or altered GAP SH3 binding protein, nucleolin, Vesicle Associated Protein 1, or DIS gene, or any protein encoded by an oncogene, or by an inactive tumor suppressor gene, may also be used.

One skilled in the art will appreciate that a promoter is chosen that directs expression of the chosen gene in the tissue in which cancer is expected to develop. For example, as noted above, any promoter that promotes expression of Taz in blood cells can be used in the expression constructs of the present invention. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is desirable, however, that an intact region of the transcriptional regulatory elements of a gene is used. Once a suitable transgene construct has been made, any suitable technique for introducing this construct into embryonic cells can be used.

Animals suitable for transgenic experiments can be obtained from standard commercial sources such as Taconic (Germantown, N.Y.). Many strains are suitable, but Swiss Webster (Taconic) female mice are desirable for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a transgenic mouse or rat. An example of a protocol that can be used to produce a transgenic animal is provided below.

Production of Transgenic Mice and Rats

The following is but one desirable means of producing transgenic mice. This general protocol may be modified by those skilled in the art.

Female mice six weeks of age are induced to superovulate with a 5 IU injection (0.1 cc, IP) of pregnant mare serum gonadotropin (PMSG; Sigma) followed 48 hours later by a 5 IU injection (0.1 cc, IP) of human chorionic gonadotropin (hCG, Sigma). Females are placed together with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with humidified atmosphere at 5% CO₂, 95% air until the time of injection. Embryos can be implanted at the two-cell stage.

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos are transferred. After the transferring the embryos, the incision is closed by two sutures.

A desirable procedure for generating transgenic rats is similar to that described above for mice (Hammer et al., Cell 63:1099-112 (1990). For example, thirty-day old female rats are given a subcutaneous injection of 20 IU of PMSG (0.1 cc) and 48 hours later each female placed with a proven, fertile male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO₂ asphyxiation) and their oviducts removed, placed in DPBA (Dulbecco's phosphate buffered saline) with 0.5% BSA and the embryos collected. Cumulus cells surrounding the embryos are removed with hyaluronidase (1 mg/ml). The embryos are then washed and placed in EBSs (Earle's balanced salt solution) containing 0.5% BSA in a 37.5° C. incubator until the time of microinjection.

Once the embryos are injected, the live embryos are moved to DPBS for transfer into foster mothers. The foster mothers are anesthetized with ketamine (40 mg/kg, IP) and xulazine (5 mg/kg, IP). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10 to 12 embryos are transferred into each rat oviduct through the infundibulum. The incision is then closed with sutures, and the foster mothers are housed singly.

Generation of Knockout Mice

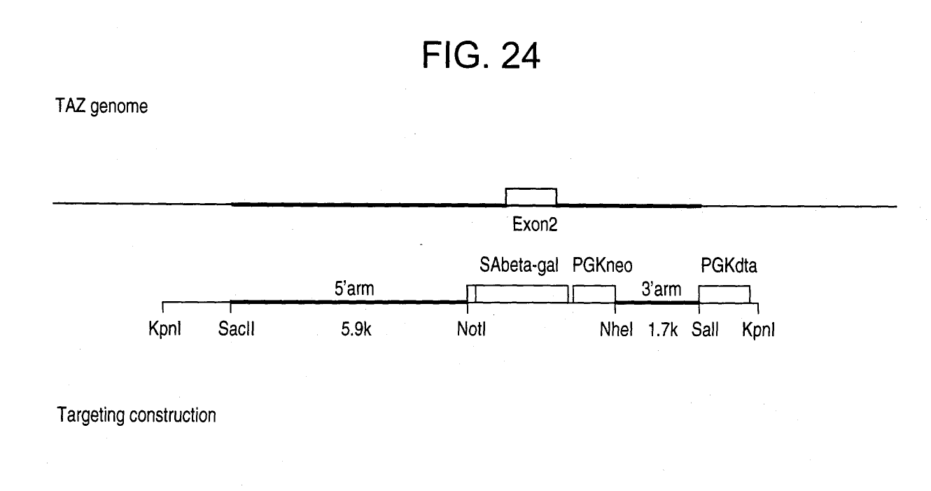

We generated TAZ knock out mice by replacing exon 2 (which encodes amino acids 1-144 of murine DIS; SEQ ID NO:2) of the mouse TAZ gene with the pSAbeta-galpGKneopGKdta positive-negative selection vector (FIG. 24). The TAZ knock out construct was transfected into an Embryonic Stem (ES) cell line and two positive ES clones were obtained and confirmed by PCR and by Southern blot. A Southern blot for the neo gene also confirmed that only exon 2 of TAZ was replaced. We performed microinjections with these ES clones and obtained chimeric mice. Nine F1 TAZ^{+/-} mice were obtained from different chimeric mice and these mice were mated to each other to generate TAZ^{-/-} knockout mice.

In addition to the particular method described above, the following is another example for the generation of a knockout mouse and the protocol may be readily adapted or modified by those skilled in the art.

ES cells, for example, 10⁷ AB1 cells, may be electroporated with 25 μg targeting construct in 0.9 ml PBS using a Bio-Rad Gene Pulser (500 μF, 230 V). The cells may then be plated on one or two 10-cm plates containing a monolayer of irradiated STO feeder cells. Twenty-four hours later, they may be subjected to G418 selection (350 μg/ml, Gibco) for 9 days. Resistant clones may then be analyzed by Southern blotting after Hind III digestion, using a probe specific to the targeting construct. Positive clones are expanded and injected into C57BL/6 blastocysts. Male chimeras may be back-crossed to C57BL/6 females. Heterozygotes may be identified by Southern blotting and intercrossed to generate homozygotes.

The targeting construct may result in the disruption of the gene of interest, e.g., by insertion of a heterologous sequence containing stop codons, or the construct may be used to replace the wild-type gene with a mutant form of the same gene, e.g., a “knock-in.” Furthermore, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, Yamamoto et al. (Cell 101:57-66 (2000)).

Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All references cited herein are hereby incorporated by reference.

We claim:

1. A method of determining the presence or absence of an alteration in the genetic material of a cell, said method comprising determining whether a cell can act as a permissive host for the replication and dissemination of a BMD-13 T-HR mutant virus, said BMD-13 T-HR mutant virus being capable of replicating and disseminating in an abnormally proliferating cell and not being capable of replicating and disseminating in a normal cell.

2. The method of claim 1, wherein the presence of said alteration in the genetic material is indicative of an organism carrying this genetic alteration having, or being at an increased risk of developing, a proliferative disease.

3. The method of claim 1, wherein said cell is determined to have an alteration in a Taz, a GAP SH3 Binding Protein, a nucleolin, a Vesicle Associated Protein 1, or a Death Inducer with SAP Domain nucleic acid sequence.

4. The method of claim 1, wherein said cell is determined to have an alteration in a Taz, a GAP SH3 binding protein, a Nucleolin, a Vesicle Associated Protein 1, or a Death Inducer with SAP Domain polypeptide.

5. The method of claim 1, wherein said BMD-13 T-HR mutant virus contains an alteration at the second position of the amino acid sequence of a polyoma T antigen.

6. The method of claim 5, wherein said alteration at said second position of the amino acid sequence of a polyoma T antigen is an Aspartic Acid to Asparagine substitution.

7. The method of claim 1, wherein said BMD-13 T-HR mutant virus contains an alteration comprising the deletion of amino acids 2 to 4 of a polyoma T antigen.

8. A method of killing an abnormally proliferating cell, said method comprising the steps of:
(a) contacting an abnormally proliferating cell with a T-HR mutant specific for a cell carrying a Taz, a GAP SH3 binding protein, a nucleolin, a Vesicle Associated Protein 1, or a Death Inducer with SAP Domain alteration; and
(b) allowing said T-HR mutant to lyse said cell.

9. The method of claim 8, wherein said T-HR mutant is the BMD-13 T-HR mutant virus.

10. The method of claim 9, wherein said BMD-13 T-HR mutant virus contains an alteration at the second position of the amino acid sequence of a polyoma T antigen.

11. The method of claim 10, wherein said alteration is an Aspartic Acid to Asparagine substitution.

12. The method of claim 9, wherein said BMD-13 T-HR mutant virus contains an alteration comprising the deletion of amino acids 2 to 4 of a polyoma T antigen.

13. The method of claim 8, wherein said T-HR mutant is a virus selected from the group consisting of, simian virus 40, human polyoma virus, murine polyoma virus, herpes virus, primate adenoviruses, parvovirus, and papilloma virus.

14. A BMD-13 T-HR mutant virus.

15. The BMD-13 T-HR mutant virus of claim 14, wherein said BMD-13 T-HR mutant virus contains an alteration at the second position of the amino acid sequence of a polyoma T antigen.

16. The BMD-13 T-HR mutant virus of claim 15, wherein said alteration is an Aspartic Acid to Asparagine substitution.

17. The BMD-13 T-HR mutant virus of claim 14, wherein said BMD-13 T-HR mutant virus contains an alteration comprising the deletion of amino acids 2 to 4 of a polyoma T antigen.

18. An isolated nucleic acid encoding a Death Inducer with SAP Domain amino acid sequence, wherein said Death Inducer with SAP Domain amino acid sequence is at least 30% identical to the amino acid sequence of SEQ ID NO:2 or SEQ ID NO:4 and induces DNA condensation and apoptosis in a mammalian cell.

19. The nucleic acid of claim 18, wherein said Death Inducer with SAP Domain amino acid sequence comprises the amino acid sequence of SEQ ID NO:2.

20. The nucleic acid of claim 18, wherein said Death Inducer with SAP Domain amino acid sequence comprises the amino acid sequence of SEQ ID NO:4.

21. A method of killing an abnormally proliferating cell, said method comprising the step of contacting said abnormally proliferating cell with a Death Inducer with SAP Domain nucleic acid sequence, wherein said contacting results in the expression of a Death Inducer with SAP Domain polypeptide in said abnormally proliferating cell.

22. The method of claim 21, wherein said Death Inducer with SAP Domain nucleic acid sequence comprises the nucleic acid sequence of SEQ ID NO:1 or SEQ ID NO:3.

23. The method of claim 21, wherein said abnormally proliferating cell is an endometrial, prostate, or ovarian cell.

24. A method of identifying a mammal having or at increased risk of acquiring a proliferative disease, said method comprising the step of determining whether there is a loss of heterozygosity in a Death Inducer with SAP Domain nucleic acid of said mammal, wherein said loss of heterozygosity in a Death Inducer with SAP Domain nucleic acid is indicative of said mammal having, or being at increased risk of acquiring a proliferative disease.

25. The method of claim 24, wherein said method is for identifying a mammal having a proliferative disease.

26. The method of claim 24, wherein said method is for identifying a mammal at increased risk of acquiring a proliferative disease.

27. The method of claim 24, wherein said determining is done by polymerase chain reaction amplification, single nucleotide polymorphism determination, restriction fragment length polymorphism analysis, hybridization analysis, or mismatch detection analysis.

28. A method of decreasing proliferation of an abnormally proliferating cell, said method comprising the step of contacting said abnormally proliferating cell with a Taz nucleic acid sequence, wherein said contacting results in the expression of a Taz polypeptide having wild-type activity in said abnormally proliferating cell.

29. A method of decreasing virus replication and dissemination, said method comprising the step of contacting a cell infected with a virus with a T-HR mutant target gene nucleic acid sequence, wherein said contacting results in the expression of a T-HR mutant target gene encoded polypeptide in said cell infected with said virus and prevents said virus from replicating and disseminating.

30. The method of claim 29, wherein said T-HR mutant target gene nucleic acid sequence is a Taz, a GAP SH3 binding protein, a nucleolin, a Vesicle Associated Protein 1, or a Death Inducer with SAP Domain nucleic acid sequence.

31. A knockout mouse comprising a knockout mutation in a genomic Death Inducer with SAP Domain nucleic acid sequence.

32. A transgenic mouse whose genome comprises a nucleic acid construct including a Death Inducer with SAP Domain nucleic acid sequence, which is operably linked to transcriptional regulatory elements and encodes a Death Inducer with SAP Domain polypeptide.

33. The transgenic mouse of claim 32, wherein said Death Inducer with SAP Domain polypeptide is mutant.

34. The transgenic mouse of claim 32, wherein said transcriptional regulatory elements include a promoter that is a tissue-specific promoter.

35. A cell line derived from cells isolated from said transgenic mouse of claim 32.

36. A method of identifying a compound which modulates cell proliferation, the method comprising:
a) exposing a cell or a cell extract to a test compound, and
b) measuring whether said test compound modulates Taz, Nucleolin, Vesicle Associated Protein 1, or Death Inducer with SAP Domain levels, relative to Taz, Nucleolin, Vesicle Associated Protein 1, or Death Inducer with SAP Domain levels in a cell or cell extract not exposed to said test compound.

37. The method of claim 36, wherein said Taz, Nucleolin, Vesicle Associated Protein 1, or Death Inducer with SAP Domain is a Taz, Nucleolin, Vesicle Associated Protein 1, or Death Inducer with SAP Domain polypeptide.

38. The method of claim 36, wherein said Taz, Nucleolin, Vesicle Associated Protein 1, or Death Inducer with SAP Domain is a Taz, Nucleolin, Vesicle Associated Protein 1, or Death Inducer with SAP Domain nucleic acid.

39. The method of claim 36, wherein said compound may be used to treat a proliferative disease.

40. The method of claim 39, wherein said proliferative disease is due to a proliferative disease-associated alteration in a Taz, Nucleolin, Vesicle Associated Protein 1, or Death Inducer with SAP Domain nucleic acid sequence.

41. The method of claim 39, wherein said proliferative disease is cancer.

42. The method of claim 41, wherein said cancer is leukemia or ovarian cancer.


SEQUENCE LISTING (4 sequences)

SEQ ID NO: 1 - DNA, 3869 bp, Mus musculus
ggggggtttgaaatggcttctcggttaacccgggccagactcaggtatctgctatagaagggaaacaagtgaaagttttcccccccttgcatcatggctcagtttggaggacagaagaatccaccatgggctactcagtttacagccactgcggtctcacaaccagctgcactaggtgttcagcagccatcacttctgggagcatctcctaccatttatacccagcagactgcattggcggcggcaggccttaccacacaaacgccagcaaactatcagttaacacaaactgcggcactgcagcaacaagctgcagctgtattacagcagcaatattcacaacctcagcaggccttgtatagtgtgcagcagcagttgcaacaacctcagcagaccattttaacacagccagctgttgcattgcccacaagccttagcctgtcgactcctcagcctgcagcacagattactgtatcatatccaacaccaaggtccagtcaacagcaaactcaacctcagaagcagcgtgttttcacaggagtggttacaaagctacatgatacatttggatttgtggatgaagatgtattctttcagcttggtgctgttaaagggaaaaccccccaagttggtgatagagtattggttgaagcaacttataatcctaatatgccttttaaatggaatgcacaaagaattcaaacactaccaaatcagaatcagtctcaaacgcaacctttactgaagactccgactgctgttattcagccgattgtgccacagacaacgtttggtgttcaggcacagccccaaccccagtcattattgcaggcccagatctcagctgcctctattacaccactattgcagacgcagccacagcccttattacagcagccacagcagaaagctggtttattgcagcctcctgtccgaatagtgtcacagccacaacctgcgcggagattagatccaccatcacgattttcaggaagaaacgacagaggggatcaagtacctaatagaaaagatgaccgaagtcgtgaaagggacagagaaagacgcagatctagagaaagatcacctcagaggaaacgttcccgggagaggtcaccccggagagaaagagagcgctcccctcggagagtccgtcgtgtcgttccacggtacacagtgcagttttcaaagttttctttagattgtcccagttgtgacatgatggaactaaggcgccgttatcagaacttatatattcctagtgacttttttgatgctcagtttacatgggtggatgctttccctttgtcaagaccatttcaactgggaaattactgcaatttttatgtgatgcaccgagaagtagagtccttagaaaaaaatatggctgttcttgatccacctgatgctgaccacctgtacagtgcaaaggtaatgctgatggctagccctagtatggaagacttgtatcataagtcatgtgctcttgctgaagacccacaagaccttcgtgatggttttcagcatcctgctagacttgttaagtttctagtgggaatgaaaggcaaggatgaagccatggccattggaggccactggtctccttcgctggatggaccaaacccagaaaaagatccctctgtgttgattaaaactgccattcgttgttgtaaggctctgacaggcattgatctaagtgtatgcacacagtggtaccgttttgcagagattcgctaccatcgccctgaggagacccacaaggggcgtacagttccagctcatgtggagacagtggttttatttttcccggatgtttggcattgccttcccacccgctcagagtgggaaaccctctcccgaggatacaagcagcagctggtcgagaagcttcagggtgaacgcaagaaggctgatggagaacaggatgaagaagagaaggatgatggtgaagttaaagagatcgccactcctacccattggtctaagcttgatccaaaggcaatgaaggtaaatgatctccgaaaagaattagaaagtcgagctctcagttccaaaggactaaaatcgcagttaatagctcgcctaacaaagcagcttaaaatagaagaacaaaaagaagagcagaaggaattagagaagtctgaaaaggaagaggaagatgaggatgataagaagtctgaggatgataaagaggaagaagaaagaaaacgtcaagaagaagtggaacgacagcgtcaagaaagaagatacattttgcctgatgaacctgccataattgtgcatccgaactgggctgcaaaaagtggcaagtttgattgcagcatcatgtctttgagtgtccttttggattacagattggaagataataaagaacattcttttgaggtttcactgtttgcagaacttttcaatgaaatgcttcaaagagactttggggttagaatatacaaatcattactctctcttcctgagaaagaggacaaaaaagataaggagaagaaaagcaaaaaagaagagagaaaagataaaaaagaagaaagagaagatgatattgatgaaccaaaaccaaaacggagaaaatcaggcgacgataaagacaaaaaagaagacagagatgagagaaagaaagaagaaaaaagaaaagatgattctaaagatgatgatgaaactgaagaagataacaatcaagatgagtatgacccaatggaggcagaggaagctgaggatgaagatgacgatagggaggaggaggaagtaaaacgagatgacaaaagggatgtcagccggtactgcaaggacagacctgcgaaagataaggaaaaagagaagcctcaaatggtcacagttaacagggatctgctaatggcctttgtttattttgatcaaagtcattgcggttaccttcttgaaaaggatttggaagaaatactatatactcttggactgcatctttcacgggctcaggtaaagaaacttcttaataaagtagtactccgagaatcgtgcttttatcggaaattaacagacacctcgaaagatgatgagaaccatgaagagtcagaggcactgcaggaagacatgctaggaaacagattattacttccaacaccaacaataaaacaggaatcaaaagatggagaggaaaatgtagggcttattgtgtacaatggtgcaatggtggatgttgggagtctcctacaaaaactggaaaagagtgagaaagtaagagctgaggtggaacagaagctccagttactagaggagaaaacagatgaagatgggaaaactatattaaacttggagaactctaacaaaagcctctctggtgaacttagagaggtcaaaaaagaccttggtcaattacaagaaaacctggaggtttcagaaaacatgaatttgcaatttgaaaaccaattgaataaaacactcagaaacttatctacagttatggatgatatccacactgtcctcaaaaaggataatgtaaagagtgaagacagagatgagaaatccaaggagaacggctcaggtgtatgacacagtgcacttggggatgagtgtgttaatagtgtactataaacaaaataatcatgagatgggaatgtttcacggcagtgcatgcttgactttagtagtataaacatatatgttagttcaaatgatgtataaagttttatgaatgtgagtctgcttttgaaaattgcctgtaatttctagcattcaaattattaaatactcactgagtgaagaattttgcattgcaaaaccttttaggatgaacttggttatagtttccccaataaagttcatcagtgtcattgacaatgacaagtaattaaaaccaaaaaaaaaaaaaacaaacaccaaccagg

SEQ ID NO: 2 - protein, 1146 aa, Mus musculus
MAQFGGQKNPPWATQFTATAVSQPAALGVQQPSLLGASPTIYTQQTALAAAGLTTQTPANYQLTQTAALQQQAAAVLQQQYSQPQQALYSVQQQLQQPQQTILTQPAVALPTSLSLSTPQPAAQITVSYPTPRSSQQQTQPQKQRVFTGVVTKLHDTFGFVDEDVFFQLGAVKGKTPQVGDRVLVEATYNPNMPFKWNAQRIQTLPNQNQSQTQPLLKTPTAVIQPIVPQTTFGVQAQPQPQSLLQAQISAASITPLLQTQPQPLLQQPQQKAGLLQPPVRIVSQPQPARRLDPPSRFSGRNDRGDQVPNRKDDRSRERDRERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAVLDPPDADHLYSAKVMLMASPSMEDLYHKSCALAEDPQDLRDGFQHPARLVKFLVGMKGKDEAMAIGGHWSPSLDGPNPEKDPSVLIKTAIRCCKALTGIDLSVCTQWYRFAEIRYHRPEETHKGRTVPAHVETVVLFFPDVWHCLPTRSEWETLSRGYKQQLVEKLQGERKKADGEQDEEEKDDGEVKEIATPTHWSKLDPKAMKVNDLRKELESRALSSKGLKSQLIARLTKQLKIEEQKEEQKELEKSEKEEEDEDDKKSEDDKEEEERKRQEEVERQRQERRYILPDEPAIIVHPNWAAKSGKFDCSIMSLSVLLDYRLEDNKEHSFEVSLFAELFNEMLQRDFGVRIYKSLLSLPEKEDKKDKEKKSKKEERKDKKEEREDDIDEPKPKRRKSGDDKDKKEDRDERKKEEKRKDDSKDDDETEEDNNQDEYDPMEAEEAEDEDDDREEEEVKRDDKRDVSRYCKDRPAKDKEKEKPQMVTVNRDLLMAFVYFDQSHCGYLLEKDLEEILYTLGLHLSRAQVKKLLNKVVLRESCFYRKLTDTSKDDENHEESEALQEDMLGNRLLLPTPTIKQESKDGEENVGLIVYNGAMVDVGSLLQKLEKSEKVRAEVEQKLQLLEEKTDEDGKTILNLENSNKSLSGELREVKKDLGQLQENLEVSENMNLQFENQLNKTLRNLSTVMDDIHTVLKKDNVKSEDRDEKSKENGSGV

SEQ ID NO: 3 - DNA, 3856 bp, Homo sapiens
gaagttggcgcatgcgcctaaagctgacgggtttgaaatggcttcgatgttagccgggacccgactcagatcgatgctatagaagacaaacaaggaaaggttttttttccttttgcatcatggctcaatttggaggacagaagaatccgccatgggctactcagtttacagccactgcagtatcacagccagctgcactgggtgttcaacagccatcactccttggagcatctcctaccatttatacacagcaaactgcattggcagcagcaggccttaccacacaaactccagcaaactatcagttaacacaaactgctgcattgcagcaacaagccgcagctgcagcagctgcattacaacagcaatattcacaacctcagcaggccctgtatagtgtgcaacaacagttacagcaaccccagcaaaccctcttaacacagccagctgttgcactgcctacaagccttagcctgtctactcctcagccaacagcacaaataactgtatcatatccaacaccaaggtccagtcaacagcaaacccagcctcagaagcagcgtgttttcacaggggtggttacaaaactacatgatacatttggatttgtggatgaagatgtattctttcagcttagtgctgtcaaagggaaaaccccccaagtaggtgacagagtattggttgaagctacttataatcctaatatgccttttaaatggaatgcacagagaattcaaacactaccaaatcagaatcagtcgcaaacccagccattactgaagactcctcctgctgtacttcagccaattgcaccacagacaacatttggtgttcagactcagccccagccccagtcactgctgcaggcacagatttcagcagcttctattacaccactattgcagactcaaccacagcccttattacagcagcctcagcaaaaagctggtttattgcagcctcctgttcgtatagtttcacagccacaaccggcacgacgattagatcccccatcccgattttcaggaagaaatgacagaggggatcaagtgcctaacagaaaagatgatcgaagtcgtgagagagagagagaaagacgtagatcgagagaaagatcacctcagaggaaacgttcccgggaaagatctccacgaagagagcgagagcgatcacctcggagagttcgacgtgttgttccacgttacacagttcagttttcaaagttttctttagattgtcccagttgtgacatgatggaactaaggcgccgttatcaaaatttgtatatacctagtgacttttttgatgctcaatttacatgggtggatgctttccctttgtcaagaccatttcagctgggaaattactgcaatttttatgtaatgcacagagaagtagagtccttagaaaaaaatatggccattcttgatccaccagatgctgaccacttatacagtgcaaaggtaatgctgatggctagccctagtatggaagatttatatcataagtcatgtgctcttgctgaggacccacaagaacttcgagatggattccaacatcctgctagacttgttaagtttttagtgggcatgaaaggcaaggatgaagctatggccattggaggccactggtctccttcgttggatggaccagacccagaaaaagatccctctgtgttgattaagactgctattcgttgttgtaaggctctgacaggcattgatctaagtgtgtgcacacaatggtaccgttttgcagagattcgctaccatcgccctgaggagacccacaaggggcgtacagttccagctcatgtggagacagtggttttatttttcccggatgtttggcattgccttcccacccgctcagagtgggaaaccctctcccgaggatacaagcagcagctggtcgagaagcttcagggtgaacgcaaggaggctgatggagaacaggatgaagaagagaaggatgatggtgaagctaaagaaatttctacacctacccattggtctaaacttgatccaaagacaatgaaggtaaatgacctccgaaaagaattagaaggtcgagctcttagttccaaaggattaaaatcccagttaatagcccgattgacaaaacagcttaaagtagaggaacaaaaagaagaacagaaggagttagagaaatctgaaaaagaagaggatgaggatgatgataggaaatctgaagacgataaagaggaagaagaaaggaaacgtcaagaggaaatagaacgccagcgtcgagaaagaagatatattttgcctgatgaaccggccatcattgtacatccaaattgggctgcaaaaagtggcaagtttgattgtagcatcatgtctttgagtgtcctattggactacagattagaggataataaagaacattcatttgaggtttcattgtttgcggaacttttcaacgaaatgcttcaaagagattttggtgtccgtatatacaaatcattactgtctcttcctgagaaagaggacaaaaaagaaaaggataaaaaaagcaaaaaagatgagagaaaagataaaaaagaagaaagagatgatgaaactgatgaaccaaaacccaaacggagaaaatcaggcgatgataaagataaaaaagaagatagagatgaaaggaagaaagaagataaaagaaaaggtgattctaaagatgatgatgaaactgaagaagataacaatcaagatgaatatgaccctatggaagcagaagaagctgaggatgaagaagatgatagggatgaggaagaaatgaccaaacgagatgacaaaagagatatcaacagatactgcaaggagaggccctctaaagataaggaaaaagaaaagactcaaatgatcacaattaacagagatctgttaatggcttttgtttattttgatcaaagtcattgtggttaccttcttgaaaaggatttggaagaaatactttatactcttggactacatctttctcgggctcaggtaaagaagcttcttaataaagtagtgctccgtgaatcttgcttttaccggaaattaacagacacctcaaaagatgaagagaaccatgaagagtctgagtcattgcaggaagatatgctaggaaacagattattacttccaacaccaacagtaaagcaggaatcaaaggatgtggaagaaaatgttggcctcattgtgtacaatggtgcaatggtagatgtaggaagcctcttgcaaaaattggaaaagagcgaaaaagtaagagctgaggtagaacagaagctgcagttactagaagaaaaaacagatgaagatgaaaaaaccatattaaatttggagaattccaacaaaagcctctctggtgaactcagagaagttaaaaaggaccttagtcagttacaagaaaacttaaagatttcagaaaacatgagtttacaatttgaaaaccaaatgaataagacaatcagaaacttatctacggtaatggatgaaatccacactgttctcaagaaggataatgtaaagaatgaagacaaagatcaaaaatccaaggagaatggtgccagtgtatgataaaatccatgtagtgatgaggaatggtgttaaataatgtaatatataaaaatcatgatataagaatgtttgaaggtgatgcatgtttgattttagtagtataaatgtattttagttcaaatgatgtataaagttttatgaatgtgagtttctgcttttgaaaattgcttgtaattcctagccttcaaattattaaacactccttgagtgaaataattttgcattgcaaagtgttttaggatgaactttgttatagttttaactccaataaagttcatcagttt

SEQ ID NO: 4 - protein, 1150 aa, Homo sapiens
MAQFGGQKNPPWATQFTATAVSQPAALGVQQPSLLGASPTIYTQQTALAAAGLTTQTPANYQLTQTAALQQQAAAAAAALQQQYSQPQQALYSVQQQLQQPQQTLLTQPAVALPTSLSLSTPQPTAQITVSYPTPRSSQQQTQPQKQRVFTGVVTKLHDTFGFVDEDVFFQLSAVKGKTPQVGDRVLVEATYNPNMPFKWNAQRIQTLPNQNQSQTQPLLKTPPAVLQPIAPQTTFGVQTQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQDEEEKDDGEAKEISTPTHWSKLDPKTMKVNDLRKELEGRALSSKGLKSQLIARLTKQLKVEEQKEEQKELEKSEKEEDEDDDRKSEDDKEEEERKRQEEIERQRRERRYILPDEPAIIVHPNWAAKSGKFDCSIMSLSVLLDYRLEDNKEHSFEVSLFAELFNEMLQRDFGVRIYKSLLSLPEKEDKKEKDKKSKKDERKDKKEERDDETDEPKPKRRKSGDDKDKKEDRDERKKEDKRKGDSKDDDETEEDNNQDEYDPMEAEEAEDEEDDRDEEEMTKRDDKRDINRYCKERPSKDKEKEKTQMITINRDLLMAFVYFDQSHCGYLLEKDLEEILYTLGLHLSRAQVKKLLNKVVLRESCFYRKLTDTSKDEENHEESESLQEDMLGNRLLLPTPTVKQESKDVEENVGLIVYNGAMVDVGSLLQKLEKSEKVRAEVEQKLQLLEEKTDEDEKTILNLENSNKSLSGELREVKKDLSQLQENLKISENMSLQFENQMNKTIRNLSTVMDEIHTVLKKDNVKNEDKDQKSKENGASV